(19) United States
(12) Patent Application Publication
(10) Pub. No.: US 2009/0005575 A1
(43) Pub. Date: Jan. 1, 2009

(54) Preparation of diazapentalene derivatives via epoxydation of dihydropyrroles

(75) Inventors: Martin Quibell, Cambridge (GB); Yikang Wang, Cambridge (GB); James Nally, Cambridge (GB); John Paul Watts, Cambridge (GB); Virendar Kumar Agrawal, Ahmedabad (IN); Michael Standen, Albany, OR (US)

Correspondence Address: Edwards Angell Palmer & Dodge LLP, P.O. Box 55874, Boston, MA, 02205, US

(73) Assignee: Amura Therapeutics Ltd., Cambridge (GB)

(21) Appl. No.: 12/069,422

(22) Filed: Feb. 8, 2008

Related U.S. Application Data
(63) Continuation of application No. PCT/GB2006/003061, filed on Aug. 10, 2006.
(60) Provisional application No. 60/707,072, filed on Aug. 10, 2005.

(51) Int. Cl. C07D 487/04

(52) U.S. Cl. 548/453

(57) ABSTRACT

The present invention relates to a process for preparing a compound of formula I,

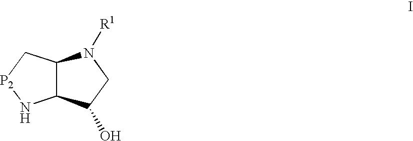

wherein
R¹ is Pg¹ or P₁′;
P₁′ is CO-hydrocarbyl;
P₂ is CH₂, O or N-Pg²; and
Pg¹ and Pg² are each independently nitrogen protecting groups;
(i) reacting a compound of formula II with a dioxirane to form an epoxide of formula III;

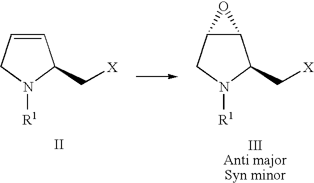

where X is selected from CN, CH₂N₃, CH₂NH-Pg², ONH-Pg², NHNH-Pg², N(Pg²)NH-Pg²;
(ii) converting a compound of formula III to a compound of formula I

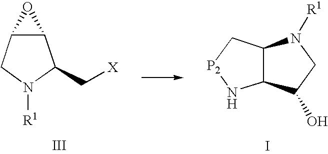

The present invention relates to a process for preparing 5,5-bicyclic building blocks that are useful in the preparation of cysteinyl proteinase inhibitors, especially CAC1 inhibitors.

### BACKGROUND TO THE INVENTION

Proteinases participate in an enormous range of biological processes and constitute approximately 2% of all the gene products identified following analysis of several completed genome sequencing programmes. Proteinases mediate their effect by cleavage of peptide amide bonds within the myriad of proteins found in nature.

This hydrolytic action involves recognising, and then binding to, specific three-dimensional electronic surfaces of a protein, which aligns the bond for cleavage precisely within the proteinase catalytic site. Catalytic hydrolysis then commences through nucleophilic attack of the amide bond to be cleaved either via an amino acid side-chain of the proteinase itself or through the action of a water molecule that is bound to and activated by the proteinase.

Proteinases in which the attacking nucleophile is the thiol side-chain of a Cys residue are known as cysteine proteinases. The general classification of “cysteine proteinase” contains many members found across a wide range of organisms from viruses, bacteria, protozoa, plants and fungi to mammals.

Cysteine proteinases are classified into “clans” based upon similarity of their three-dimensional structure or a conserved arrangement of catalytic residues within the proteinase primary sequence. Additionally, “clans” may be further classified into “families” in which each proteinase shares a statistically significant relationship with other members when comparing the portions of amino acid sequence which constitute the parts responsible for the proteinase activity (see Barrett A. J et al, in ‘Handbook of Proteolytic Enzymes’, Eds. Barrett, A. J., Rawlings, N. D., and Woessner, J. F. Publ. Academic Press, 1998, for a thorough discussion).

To date, cysteine proteinases have been classified into five clans, CA, CB, CC, CD and CE (Barrett, A. J. et al, 1998). A proteinase from the tropical papaya fruit ‘papain’ forms the foundation of clan CA, which currently contains over eighty distinct entries in various sequence databases, with many more expected from the current genome sequencing efforts.

Over recent years, cysteinyl proteinases have been shown to exhibit a wide range of disease-related biological functions. In particular, proteinases of the clan CA/family C1 (CAC1) have been implicated in a multitude of disease processes [a) Lecaille, F. et al, Chem. Rev. 2002, 102, 4459; (b) Chapman, H. A. et al, Annu. Rev. Physiol. 1997, 59, 63; Barrett, A. J. et al, Handbook of Proteolytic Enzymes; Academic: New York, 1998]. Examples include human proteinases such as cathepsin K (osteoporosis), cathepsins S and P (autoimmune disorders), cathepsin B (tumour invasion/metastases) and cathepsin L (metastases/autoimmune disorders), as well as parasitic proteinases such as falcipain (malaria parasite Plasmodium falciparum), cruzipain (Trypanosoma cruzi infection) and the CPB proteinases associated with Leishmaniasis [Lecaille, F. et al, ibid, Kaleta, J., ibid].

The inhibition of cysteinyl proteinase activity has evolved into an area of intense current interest [(a) Otto, H.-H. et al, Chem. Rev. 1997, 97, 133; (b) Heranandez, A A. et al, Curr. Opin. Chem. Biol. 2002, 6, 459; (c) Veber, D. F. et al, Cur. Opin. Drug Disc. Dev. 2000, 3, 362-369; (d) Leung-Toung, R. et al, Curr. Med. Chem. 2002, 9, 979]. Selective inhibition of any of these CAC1 proteinases offers enormous therapeutic potential and consequently there has been a concerted drive within the pharmaceutical industry towards the development of compounds suitable for human administration [for example, see (a) Bromme, D. et al, Curr. Pharm. Des. 2002, 8, 1639-1658; (b) Kim, W. et al, Expert Opin. Ther. Patents 2002, 12(3), 419). To date, these efforts have primarily focused on low molecular weight substrate based peptidomimetic inhibitors, the most advanced of which are in early clinical assessment.

Cysteinyl proteinase inhibitors investigated to date include peptide and peptidomimetic nitriles (e.g. see WO 03/041649), linear and cyclic peptide and peptidomimetic ketones, ketoheterocycles (e.g. see Veber, D. F. et al, Curr. Opin. Drug Discovery Dev., 3(4), 362-369, 2000), monobactams (e.g. see WO 00/59881, WO 99/48911, WO 01/09169), α-ketoamides (e.g. see WO 03/013518), cyanoamides (WO 01/077073, WO 01/068645), dihydropyrimidines (e.g. see WO 02/032879) and cyano-aminopyrimidines (e.g. see WO 03/020278, WO 03/020721).

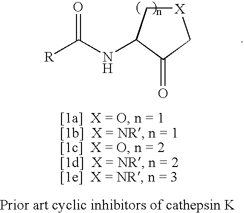

The initial cyclic inhibitors of GSK were based upon potent, selective and reversible 3-amido-tetrahydrofuran-4-ones, [1a], 3-amidopyrrolidin-4-ones [1b], 4-amido-tetrahydropyran-3-ones [1c], 4-amidopiperidin-3-ones [1d] and 4-aridoazepan-3-ones [1e] (shown above) [see (a) Marquis, R. W. et al, J. Med. Chem. 2001, 44, 725, and references cited therein; (b) Marquis, R W. et al, J. Med. Chem. 2001, 44, 1380, and references cited therein].

Further studies revealed that cyclic ketones [1], in particular the five-membered ring analogues [1a] and [1b], suffered from configurational instability due to facile epimerisation at the centre situated a to the ketone [Marquis, R. W. et al, J. Med. Chem. 2001, 44, 1380; Fenwick, A. E. et al, J. Bioorg. Med. Chem. Lett. 2001, 11, 199; WO 00/69855]. This precluded the pre-clinical optimisation of inhibitors of formulae [1a-d] and led to the development of the configurationally stable azepanone series [1e]. As an alternative to the ring expansion approach, alkylation of the α-carbon removes the ability of cyclic ketones [1] to undergo (α-enolisation and hence leads to configurational stability. However, studies have shown that α-methylation in the 3-amidopyrrolidin-4-one [1b] system results in a substantial loss in potency versus cathepsin K from K_{i,app}≈0.18 to 50 nM.

More recent studies have investigated 5,5-bicyclic systems as inhibitors of CAC1 proteinases, for example, N-(3-oxo-hexahydrocyclopenta[b]furan-3α-yl)acylamide bicyclic ketones [2] [(a) Quibell, M.; Ramjee, M. K., WO 02/57246; (b) Watts, J. et al, Bioorg. Med. Chem. 12 (2004), 2903-2925], tetaydrofuro[3,2-b]pyrrol-3-one based scaffolds [3] [Quibell, M. et al, Bioorg. Med. Chem. 12 (2004), 5689-5710], cis-6-oxohexahydro-2-oxa-1,4-diazapentalene and cis-6-oxo-hexahydropyrrolo[3,2-c]pyrazole based scaffolds 14] [Wang, Y. et al, Bioorg. Med. Chem. Lett. 15 (2005), 1327-1331], and cis-hexahydropyrrolo[3,2-b]pyrrol-3-one based scaffolds [5] [Quibell, M. et al, Bioorg. Med. Chem. 13 (2005), 609-625].

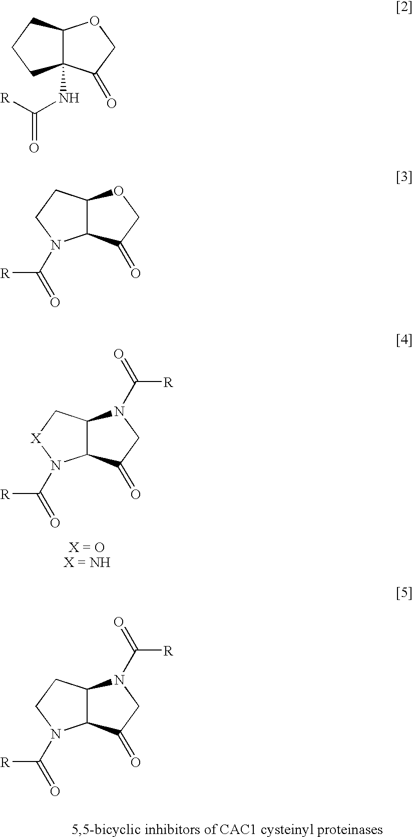

Studies have shown that the above-described 5,5-bicyclic systems exhibit promising potency as inhibitors of a range of therapeutically attractive mammalian and parasitic CAC1 cysteinyl proteinase targets. Moreover, the 5,5-bicyclic series are chirally stable due to a marked energetic preference for a cis-fused rather than a trans-fused geometry. This chiral stability provides a major advance when compared to monocyclic systems that often show limited potential for preclinical development due to chiral instability.

The present invention seeks to provide an improved process for synthesising a 5,5-bicyclic building block useful in the preparation of cysteinyl proteinase inhibitors.

More particularly, the invention seeks to provide an improved process for synthesising a cis-hexahydropyrrolo[3,2-b]pyrrol-3-one core.

Aspects of the invention are set forth below and in the accompanying claims.

### STATEMENT OF INVENTION

A first aspect of the invention relates to a process for preparing a compound of formula I, or a pharmaceutically acceptable salt thereof,

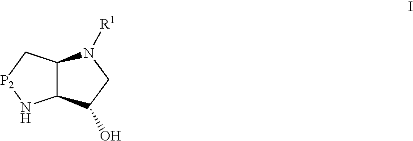

wherein

R¹ is Pg¹ or P₁′;

P₁′ is CO-hydrocarbyl;

P₂ is CH₂, O or N-Pg²; and

Pg¹ and Pg² are each independently nitrogen protecting groups; said process comprising the steps of:
(i) reacting a compound of formula II with a dioxirane to form an epoxide of formula III;

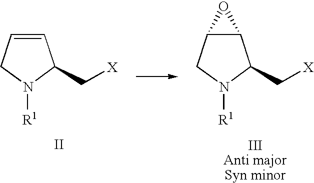

where X is selected from CN, CH₂N₃, CH₂NH-Pg², ONH-Pg², NHNH-Pg², N(Pg²)NH-Pg²;
(ii) converting a compound of formula III to a compound of formula I

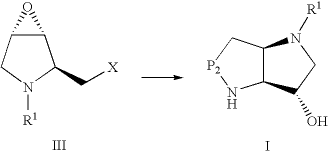

Another aspect of the invention relates to a method for preparing a cysteinyl proteinase inhibitor which comprises the above-described process.

Further aspects of the invention relate to methods of preparing compounds of formula VII, VIII and IX, where R^{x}, R^{y}, R^{w}, R^{z}, U, V, W, X′, Y, n, m, o, P₂, P_{2′} and R^{1′} are as defined in the detailed description below,

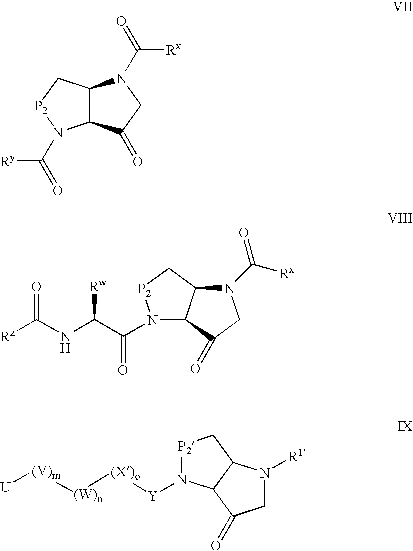

wherein said methods comprise a process according to the first aspect of the invention as set forth above.

### DETAILED DESCRIPTION

As used herein, the term “hydrocarbyl” refers to a group comprising at least C and H. If the hydrocarbyl group comprises more than one C then those carbons need not necessarily be linked to each other. For example, at least two of the carbons may be linked via a suitable element or group. Thus, the hydrocarbyl group may contain heteroatoms. Suitable heteroatoms will be apparent to those skilled in the art and include, for instance, sulphur, nitrogen, oxygen, phosphorus and silicon. Where the hydrocarbyl group contains one or more heteroatoms, the group may be linked via a carbon atom or via a heteroatom to another group, i.e. the linker atom may be a carbon or a heteroatom. The hydrocarbyl group may also include one or more substituents, for example, halo, alkyl, acyl, cycloalkyl, an alicyclic group, CF₃, OH, CN, NO₂, SO₃H, SO₂NH₂, SO₂Me, NH₂, COOH, and CONH₂. Preferably, the hydrocarbyl group is an aryl, heteroaryl, alkyl, cycloalkyl, aralkyl, alicyclic or alkenyl group. More preferably, the hydrocarbyl group is an aryl, heteroaryl, alkyl, cycloalkyl, aralkyl or alkenyl group.

As used herein, the term “alkyl” includes both saturated straight chain and branched alkyl groups which may be substituted (mono- or poly-) or unsubstituted. Preferably, the alkyl group is a C₁₋₂₀ alkyl group, more preferably a C₁₋₁₅, more preferably still a C₁₋₁₂ alkyl group, more preferably still, a C₁— alkyl group, more preferably a C₁₋₃ alkyl group. Particularly preferred alkyl groups include, for example, methyl, ethyl, propyl, isopropyl, butyl, isobutyl, tert-butyl, pentyl and hexyl. Examples of suitable substituents include halo, CF₃, OH, CN, NO₂, SO₃H, SO₂NH₂, SO₂Me, NH₂, COOH, and CONH₂.

As used herein, the term “aryl” or “Ar” refers to a C₁₋₁₂ aromatic group which may be substituted (mono- or poly-) or unsubstituted. Typical examples include phenyl and naphthyl etc. Examples of suitable substituents include alkyl, halo, CF₃, OH, CN, NO₂, SO₃H, SO₂NH₂, SO₂Me, NH₂, COOH, and CONH₂.

As used herein, the term “heteroaryl” refers to a C₄₋₂ aromatic, substituted (mono- or poly-) or unsubstituted group, which comprises one or more heteroatoms. Preferred heteroaryl groups include pyrrole, indole, benzofuran, pyrazole, benzimidazole, benzothiazole, pyrimidine, imidazole, pyrazine, pyridine, quinoline, triazole, tetrazole, thiophene and furan. Again, suitable substituents include, for example, halo, alkyl, CF₃, OH, CN, NO₂, SO₃H, SO₂NH₂, SO₂Me, NH₂, COOH, and CONH₂.

As used herein, the term “cycloalkyl” refers to a cyclic alkyl group which may be substituted (mono- or poly-) or unsubstituted. Suitable substituents include, for example, halo, alkyl, CF₃, OH, CN, NO₂, SO₃H, SO₂NH₂, SO₂Me, NH₂, COOH, CONH₂ and alkoxy.

The term “cycloalkyl(alkyl)” is used as a conjunction of the terms alkyl and cycloalkyl as given above.

The term “aralkyl” is used as a conjunction of the terms alkyl and aryl as given above. Preferred aralkyl groups include CH₂Ph and CH₂CH₂Ph and the like.

As used herein, the term “alkenyl” refers to a group containing one or more carbon-carbon double bonds, which may be branched or unbranched, substituted (mono- or poly-) or unsubstituted. Preferably the alkenyl group is a C₂₋₂₀ alkenyl group, more preferably a C₂₋₁₅ alkenyl group, more preferably still a C₂₋₁₂ alkenyl group, or preferably a C₂₄ alkenyl group, more preferably a C₂₋₃ alkenyl group. Suitable substituents include, for example, alkyl, halo, CF₃, OH, CN, NO₂, SO₃H, SO₂NH₂, SO₂Me, NH₂, COOH, CONH₂ and alkoxy.

As used herein, the term “alicyclic” refers to a cyclic aliphatic group which optionally contains one or more heteroatoms and which is optionally substituted. Preferred alicyclic groups include piperidinyl, pyrrolidinyl, piperazinyl and morpholinyl. More preferably, the alicyclic group is selected from N-piperidinyl, N-pyrrolidinyl, N-piperazinyl and N-morpholinyl. Suitable substituents include, for example, alkyl, halo, CF₃, OH, CN, NO₂, SO₃H, SO₂NH₂, SO₂Me, NH₂, COOH, CONH₂ and alkoxy.

The term “aliphatic” takes its normal meaning in the art and includes non-aromatic groups such as alkanes, alkenes and alkynes and substituted derivatives thereof.

The group P₂ is defined as CH₂, O or N-Pg². In one highly preferred embodiment of the invention, P₂ is CH₂.

The group X is selected from CN, CH₂N₃, CH₂NH-Pg², ONH-Pg², NHNH-Pg² and N(Pg²)NH-Pg². In one highly preferred embodiment of the invention, X is CN.

The present invention relates to the preparation and use of all salts, hydrates, solvates, complexes and prodrugs of the compounds described herein. The term “compound” is intended to include all such salts, hydrates, solvates, complexes and prodrugs, unless the context requires otherwise.

Appropriate pharmaceutically and veterinarily acceptable salts of the compounds of general formula (I) include salts of organic acids, especially carboxylic acids, including but not limited to acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids. Salts which are not pharmaceutically or veterinarily acceptable may still be valuable as intermediates.

The invention furthermore relates to the preparation of compounds in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

As mentioned above, the present invention seeks to provide an improved process for preparing a 5,5-bicyclic building block useful in the preparation of cysteinyl proteinase inhibitors.

The key steps of the invention involve the epoxidation of an N-protected 2,5-dihydropyrrole compound (step (i)) using a dioxirane, followed by reduction (as necessary) and intramolecular cyclisation to form a cis-5,5-bicyclic ring system.

The use of dioxiranes as oxidising agents is well documented in the literature [see (a) Hodgson, D. M. et al, Synlett, 310 (2002); (b) Adam, W. et al, Acc. Chem. Res. 22, 205, (1989); (c) Yang, D. et al, J. Org. Chem., 60, 3887, (1995); (d) Mello, R. et al, J. Org. Chem., 53, 3890, (1988); (e) Curci, R. et al, Pure & Appl. Chem., 67(5), 811 (1995); (f) Emmons, W. D. et al, J. Amer. Chem. Soc. 89, (1955)].

Preferably, the dioxirane is generated in situ by the reaction of KHSO₅ with a ketone. However, step (i) can also be carried out using an isolated dioxirane, for example a stock solution of the dioxirane formed from acetone.

More preferably, the dioxirane is generated in situ using Oxone®, which is a commercially available oxidising agent containing KHSO₅ as the active ingredient.

Thus, in one preferred embodiment, step (i) of the claimed process involves the in situ epoxidation of an N-protected 2,5-dihydropyrrole compound of formula II using Oxone® (2 KHSO₅.KHSO₄.K₂SO₄) and a ketone co-reactant.

As mentioned above, the active ingredient of Oxone® is potassium peroxymonosulfate, KHSO₅ [CAS-RN 10058-23-8], commonly known as potassium monopersulfate, which is present as a component of a triple salt with the formula 2 KHSO₅.KHSO₄.K₂SO₄ [potassium hydrogen peroxymonosulfate sulfite (5:3:2:2), CAS-RN 70693-62-8; commercially available from DuPont]. The oxidation potential of Oxone® is derived from its peracid chemistry; it is the first neutralization salt of peroxymonosulfic acid H₂SO₅ (also known as Caro's acid).

K⁺—O—S(—O)₂(—OOH) Potassium Monopersulfate

Under slightly basic conditions (pH 7.5-8.0), persulfate reacts with the ketone co-reactant to form a three membered cyclic peroxide (a dioxirane) in which both oxygens are bonded to the carbonyl carbon of the ketone. The cyclic peroxide so formed then epoxidises the compound of formula II by syn specific oxygen transfer to the alkene bond.

Preferably, the ketone is of formula V

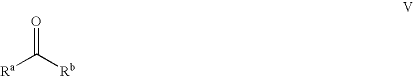

wherein R^{a} and R^{b} are each independently alkyl, aryl, haloalkyl or haloaryl.

Where R^{a} and/or R^{b} are alkyl, the alkyl group may be a straight chain or branched alkyl group. Preferably, the alkyl group is a C₁₋₂₀ alkyl group, more preferably a C₁₋₁₅, more preferably still a C₁₋₁₂ alkyl group, more preferably still, a C₁₋₄ alkyl group, more preferably a C₁₋₃ alkyl group. Particularly preferred alkyl groups include, for example, methyl, ethyl, propyl, isopropyl, butyl, isobutyl, tert-butyl, pentyl and hexyl.

As used herein, the term “haloalkyl” refers to an alkyl group as described above in which one or more hydrogens are replaced by halo.

Where R^{a} and/or R^{b} are aryl, the aryl group is typically a C₆₋₁₂ aromatic group. Preferred examples include phenyl and naphthyl etc.

As used herein, the term “haloaryl” refers to an aryl group as described above in which one or more hydrogens are replaced by halo.

By way of example, the reaction of KHSO₅ (Oxone®) with a ketone of formula V would form a dioxirane of formula VI:

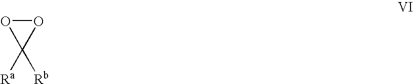

wherein R^{a} and R^{b} are as defined above.

More preferably, R^{a} and R^{b} are each independently alkyl or haloalkyl. In a highly preferred embodiment, at least one of R^{a} and R^{b} is a haloalkyl, more preferably, CF₃ or CF₂CF₃.

In one preferred embodiment, R^{a} and R^{b} are each independently methyl or trifluoromethyl.

In one preferred embodiment of the invention, the ketone is selected from acetone and a 1,1,1-trifluoroalkyl ketone.

In a more preferred embodiment of the invention, the trifluoroalkyl ketone is 1,1,1-trifluoroacetone or 1,1,1-trifluoro-2-butanone, more preferably 1,1,1-trifluoro-2-butanone.

Advantageously, epoxidation using a dioxirane leads to an increase in the ratio of anti-epoxide:syn-epoxide. By way of example, in compounds of formula III where X is CN, the use of Oxone®/1 μl, 1-trifluoro-2-butanone reagent mixtures produces >9:1 anti-epoxide:syn-epoxide mixture. Likewise, use of Oxone®/1,1,1-trifluoroacetone mixtures produces a 7:1 anti-epoxide:syn-epoxide mixture. In contrast, prior art methods for the epoxidation step using mCPBA only afford much lower anti-epoxide:syn-epoxide ratios, for example, a 2:1 ratio.

The increased ratio of anti-epoxide:syn-epoxide obtained using the conditions of the invention ultimately affords more favourable yields of the desired cis-5,5-bicyclic compound of formula I, which is formed by the subsequent intramolecular cyclisation of the anti-epoxide.

The improved selectivity ratio obtained using the process of the invention is further manifested in the fact that preferably, after extraction from the reaction medium, the resulting mixture of anti- and syn-epoxides can be enriched by trituration and/or crystallisation from organic solvents to obtain the optically pure anti-epoxide.

In one highly preferred embodiment of the invention, X is CN and said compound of formula III is purified by crystallisation to obtain the anti-epoxide in substantially pure form. In one highly preferred embodiment, the anti-epoxide is crystallised from a mixture of diethyl ether/heptane.

The rate of alkene epoxidation, together with the selectivity of reaction, the ease of extraction and the ability to obtain the pure anti-epoxide by trituration and/or recrystallation identifies the use of KHSO₅/ketone mixtures as highly advantageous reagents for the stereoselective epoxidation of compounds of formula II.

In one preferred embodiment of the invention, step (i) is carried out at a pH of about 7.5 to about 8. When dioxiranes are generated in situ, it is important to control the pH. Preferably, the pH can be controlled by using a phosphate or bicarbonate buffer.

In one preferred embodiment of the invention, step (i) is carried out in the presence of NaHCO₃.

In one preferred embodiment of the invention, step (i) is carried out using a solvent comprising acetonitrile.

In a more preferred embodiment of the invention, step (i) is carried out using a solvent comprising acetonitrile and water.

In one preferred embodiment of the invention, step (i) is carried out using a solvent mixture which further comprises a phase transfer reagent. Suitable phase transfer reagents include for example 18-crown-6 and Bu₄N⁺HSO₄⁻.

In another preferred embodiment of the invention, step (i) is carried out in a solvent mixture comprising aqueous Na₂.EDTA.

Even more preferably, step (i) is carried out using a solvent comprising acetonitrile, water and Na₂.EDTA.

In one particularly preferred embodiment of the invention, wherein R¹ is tert-butoxycarbonyl, P₂ is methylene and X is CN in said compound of formula II, step (i) is carried out using an excess of reagents in the following ratio; 1.0 equivalents of compound II, 2.0 equivalents of Oxone®, 2.0 equivalents of 1,1,1-trifluoroacetone, 1.0 equivalents of acetone, 8.6 equivalents of NaHCO₃, 0.014 equivalents of Na₂.EDTA in a mixed acetonitrile and water solvent. Preferably, the reaction is carried out at 0 to 5° C. for a reaction time of about 60 to about 90 minutes. These were found to be the optimum conditions for step (i) in the context of the present invention.

Step (ii) of the claimed process involves the intramolecular cyclisation of a compound of formula III to form a 5,5-bicyclic compound of formula I. In one preferred embodiment, the reaction proceeds via an amine intermediate of formula IV.

In one preferred embodiment, step (ii) comprises converting a compound of formula III to a compound of formula IV in situ; and converting said compound of formula IV to a compound of formula I.

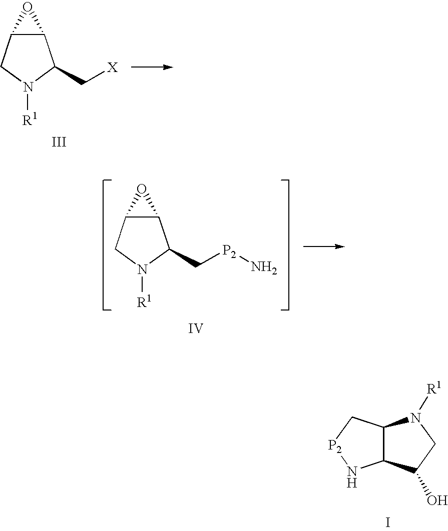

In one especially preferred embodiment, X is CN, i.e. the process involves the cyclisation of a compound of formula IIIa shown below.

Thus, in a more preferred embodiment, step (ii) comprises converting a compound of formula IIIa to a compound of formula IVa in situ; and converting said compound of formula IVa to a compound of formula Ia, (i.e. a compound of formula I wherein P₂ is CH₂).

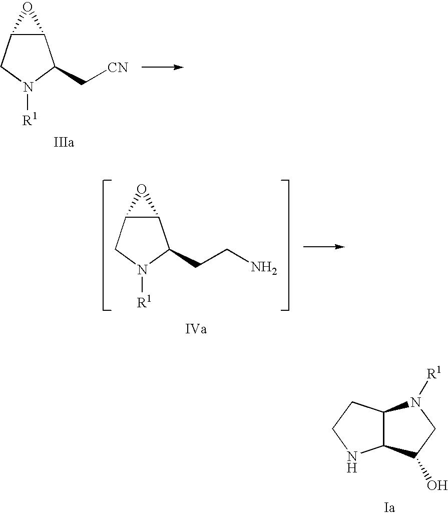

In a preferred embodiment, step (ii) comprises treating a compound of formula IIIa with sodium borohydride and cobalt (II) chloride hexahydrate. Preferably, the solvent for this step is methanol. Preferably, the reaction is carried out at ambient temperature.

In an alternative preferred embodiment, step (ii) comprises treating a compound of formula IIIa (wherein R¹ is tert-butoxycarbonyl Boc) with Raney nickel and hydrogen. Preferably, the solvent for this step is methanol containing ammonia. Preferably, the reaction is carried out at 30° C. for a reaction time of 2 hours. These conditions were found to be the optimum conditions for step (ii) in the context of the present invention in terms of yield, impurity profile and operability at scale.

In an alternative preferred embodiment, step (ii) comprises treating a compound of formula IIIa with lithium aluminium hydride in ether.

In yet another preferred embodiment, step (ii) comprises treating a compound of formula IIIa with sodium borohydride and nickel chloride.

In a preferred embodiment, said compound of formula II is of formula IIa below, and R¹ is as defined herein above, i.e. step (i) involves epoxidising a compound of formula II in which X is a cyano group to form a compound of formula IIIa

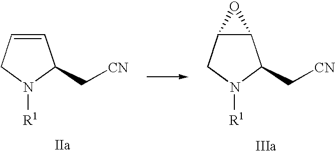

In a particularly preferred embodiment, said compound of formula IIa is prepared from a compound of formula Jib

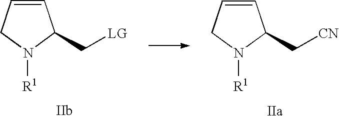

where LG is a leaving group, and R¹ is as defined above.

Preferably, the leaving group is mesylate (Ms), tosylate (Ts), OH or halo.

More preferably, said compound of formula IIa is prepared by reacting a compound of formula IIb with sodium cyanide. Preferably, the solvent is DMSO or DMF. Preferably, for this particular embodiment, the reaction is carried out at a temperature of at least about 100° C., more preferably, about 110° C. Even more preferably, said compound of formula IIa (wherein R¹ is tert-butoxycarbonyl Boc) is prepared by reacting a compound of formula IIb (wherein R¹ is tert-butoxycarbonyl Boc) with 1.5 equivalents of sodium cyanide in DMSO at 90-95° C. for 2 h. These reaction conditions were found to be the optimum conditions in the context of the present invention.

In an alternative preferred embodiment, said compound of formula IIa is prepared by reacting a compound of formula IIb with Et₄N⁺CN⁻. Preferably, for this embodiment, the reaction is carried out at a temperature of at least about 50° C., more preferably, about 60° C.

In another alternative preferred embodiment, said compound of formula IIa is prepared by reacting a compound of formula IIb with KCN, optionally in the presence of 18-crown-6.

For the embodiments using Et₄N⁺CN⁻ or KCN, preferably the solvent is DME, CHCl₃ or THF. Advantageously, these embodiments allow the reaction to be carried out at lower temperatures compared to the embodiment using sodium cyanide in DMSO or DMF.

In one preferred embodiment, the leaving group, LG, is mesylate (Ms), and said compound of formula IIb is prepared by mesylating a compound of formula IIc

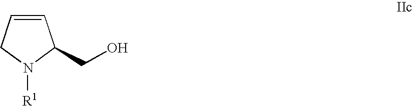

where R¹ is as defined above.

Preferably, leaving group, LG, is mesylate (Ms) and said compound of formula IIb (wherein R¹ is tert-butoxycarbonyl Boc) is prepared through the use of 1.5 equivalents mesyl chloride (MsCl) and 2.0 equivalents of triethylamine in dichloromethane. Preferably, the reaction is carried out at ambient temperature for a reaction time of 90 to 100 minutes. These conditions were found to be the optimum conditions for this step in the context of the present invention.

In an alternative preferred embodiment, the leaving group, LG, is tosylate (Ts), and said compound of formula IIb is prepared by tosylating a compound of formula IIc, where R¹ is as defined above.

In another preferred embodiment, the leaving group, LG, is OH and said compound of formula IIa is prepared by reacting a compound of formula IIc with triphenylphosphine, DEAD and acetone cyanohydrin.

In one preferred embodiment, said compound of formula IIc is prepared from a compound of formula IId

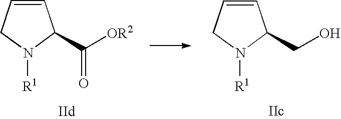

where R² is an alkyl or aryl group.

For compounds of formula IId, preferably R² is an alkyl group, more preferably methyl.

In a highly preferred embodiment, said compound of formula IIc is prepared by reacting a compound of formula IId with lithium borohydride in methanol. Preferably, the reaction is carried out at ambient temperature. Superior results were obtained using these particular reducing conditions.

In an even more highly preferred embodiment, compound of formula IIc (wherein R¹ is tert-butoxycarbonyl Boc) is prepared by reacting a compound of formula IId (wherein R¹ is tert-butoxycarbonyl Boc and R² is methyl) with 1.0 equivalent of lithium chloride, 1.0 equivalent of sodium borohydride in diethylene glycol dimethyl ether (Diglyme). Preferably, the reaction is carried out at 90-95° C. for a reaction time of 90 to 100 minutes. Superior results were also obtained using these particular reducing conditions.

In an alternative embodiment, said compound of formula IIc is prepared by reacting a compound of formula IId with lithium aluminium hydride and THF (or diethyl ether).

In one preferred embodiment, said compound of formula IId is prepared from a compound of formula IIe

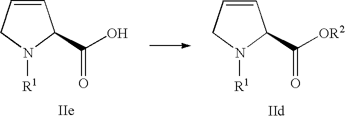

where R² is an alkyl or aryl group.

More preferably, said compound of formula IId is prepared by reacting a compound of formula IIe with (trimethylsilyl)diazomethane in toluene/MeOH. Alternative esterification conditions for this conversion will be familiar to a person having a basic knowledge of synthetic organic chemistry.

Even more preferably, said compound of formula IId (wherein R¹ is tert-butoxycarbonyl Boc and R² is methyl) is prepared by reacting a compound of formula IIe (wherein R¹ is tert-butoxycarbonyl Boc) with 3.0 equivalents of methyl iodide and 1.5 equivalents of potassium hydrogen carbonate. Preferably, the reaction is carried out in acetone at 43-45° C. for 5 to 6 hours. Superior results were obtained using these particular alkylation conditions.

The compound of formula IIe (wherein R¹ is tert-butoxycarbonyl Boc, CAS 51154-06-4) is chirally accessible at the multi-kilogram scale following a literature procedure (Sturner, R. et al, Synthesis, 1, 4648, 2001).

In an alternative preferred embodiment, said compound of formula IId is prepared by N-protecting a compound of formula IIf, or a salt thereof,

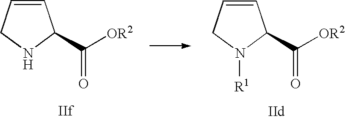

In one preferred embodiment, the nitrogen is protected by standard N-tert-butoxycarbonyl protection. Such methods will be familiar to the skilled artisan. Compound IIf, where R² is methyl, is commercially available as the HCl salt (Bachem, cat #F-1500; 2,5-dihydro-1H-pyrrole-2-carboxylic acid methyl ester).

In one embodiment, R¹ is a protecting group Pg¹ and is any nitrogen protecting group that is capable of protecting the ring nitrogen during the epoxidation step. Suitable nitrogen protecting groups will be familiar to the skilled artisan (see for example, “Protective Groups in Organic Synthesis” by Peter G. M. Wuts and Theodora W. Greene, 2^{nd} Edition). Preferred nitrogen protecting groups include, for example, tert-butyloxycarbonyl (Boc), benzyl (CBz) and 2-(biphenylyl)isopropyl. Pg² is similarly defined. Where X is N(Pg)NH-Pg², each Pg² may be the same or different.

In one highly preferred embodiment of the invention, R¹ is tert-butyloxycarbonyl (Boc).

In one especially preferred embodiment of the invention, P₂ is CH₂, X is CN and R¹ is tert-butyloxycarbonyl (Boc).

Alternatively, the R¹ group may be a P₁′ group that is compatible with the other steps of the presently claimed process, for example, a CO-hydrocarbyl group. Preferred P₁′ groups include CO-aryl, CO-aralkyl, CO-cycloalkyl, CO-alkyl and CO-alicylic group, wherein said aryl, alkyl, aralkyl, cycloalkyl and alicyclic groups are each optionally substituted by one or more substituents selected from alkyl, alkoxy, halogen, NH₂, CF₃, SO₂-alkyl, SO₂-aryl, OH, NH-alkyl, NHCO-alkyl and N(alkyl)₂.

Especially preferred P₁′ groups include CO-phenyl, CO—CH₂-phenyl and CO—(N-pyrrolidine). Additional especially preferred P₁′ groups include CO-(3-pyridyl), CO-(3-fluoro-phenyl).

In another preferred embodiment, the nitrogen protecting group R¹ is a Boc or an Fmoc group, more preferably, a Boc group.

Another preferred embodiment of the invention relates to a process as defined above which further comprises the step of protecting the free NH group of said compound of formula O. Thus, an even more preferred embodiment of the invention relates to a process as defined above which further comprises treating said compound of formula I with Fmoc-Cl and sodium carbonate in 1,4-dioxane/water mixture. This embodiment of the invention is particularly useful for the solid phase synthesis of 5,5-bicyclic systems of the invention.

A second aspect of the invention relates to a method of preparing a cysteinyl proteinase inhibitor which comprises the process as set forth above. Preferably, the cysteinyl proteinase inhibitor is a CAC1 inhibitor, more preferably a CAC1 inhibitor selected from cathepsin K, cathepsin S, cathepsin F, cathepsin B, cathepsin L, cathepsin V, cathepsin C, falcipain and cruzipain.

In yet another preferred embodiment, the process further comprises the step of converting said compound of formula I to a compound of formula VII

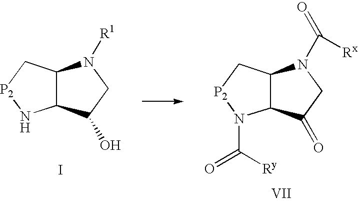

wherein R^{x} and R^{y} are each independently hydrocarbyl.

Thus, one embodiment of the invention relates to a method of preparing a cysteinyl proteinase inhibitor of formula VII, said method comprising preparing a compound of formula I as described above, and converting said compound of formula I to a compound of formula VII.

Another preferred embodiment of the invention relates to a method of preparing a cysteinyl proteinase inhibitor of formula VIII

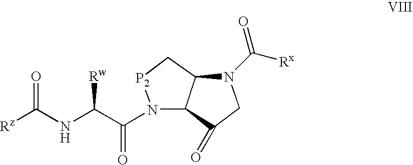

wherein

P₂ is as defined above;

R^{x} is aryl or alkyl;

R^{w} is alkyl, aralkyl, cycloalkyl(alkyl) or cycloalkyl; and

R^{z} is aryl, heteroaryl or alicyclic;

wherein said aryl, alkyl, aralkyl, cycloalkyl(alkyl), cycloalkyl, heteroaryl and alicyclic groups may be optionally substituted.

Thus, one embodiment of the invention relates to a method of preparing a cysteinyl proteinase inhibitor of formula VIII, said method comprising preparing a compound of formula I as described above, and converting said compound of formula I to a compound of formula VIII.

Another preferred embodiment of the invention relates to a method of preparing a cysteinyl proteinase inhibitor of formula VIII as shown above, wherein:

P₂ is as defined above;

R^{x} is aryl;

R^{w} is alkyl, aralkyl, cycloalkyl(alkyl); and

R^{z} is aryl or heteroaryl;

wherein said aryl, alkyl, aralkyl, cycloalkyl(alkyl) and heteroaryl groups may be optionally substituted.

Preferred substituents for said aryl, alkyl, aralkyl, cycloalkyl(alkyl) and heteroaryl groups include, for example, OH, alkyl, halo, acyl, alkyl-NH₂, NH₂, NH(alkyl), N(alkyl)₂, and an alicyclic group, wherein said alicyclic group is itself optionally substituted by one or more alkyl or acyl groups; for example the substituent is preferably a piperazinyl or piperidinyl group optionally substituted by one or more alkyl or acyl groups.

In one particularly preferred embodiment, R^{z} is an aryl or heteroaryl group optionally substituted by a piperazinyl or piperidinyl group, each of which may in turn be optionally substituted by one or more alkyl or acyl groups.

Thus, in one highly preferred embodiment, CO—R^{z} is selected from the following:

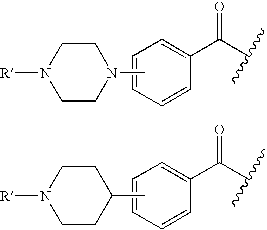

where R′ is alkyl or acyl.

In another particularly preferred embodiment, R^{z} is a 5-membered heteroaryl group or a 6-membered alicyclic group optionally substituted by one or more alkyl groups.

Thus, in another highly preferred embodiment, CO—R^{z} is selected from the following:

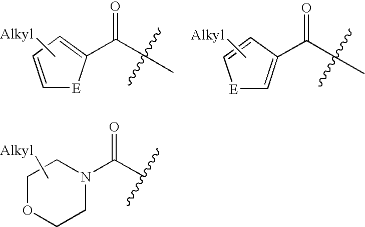

where E and alkyl are as defined herein.

Preferably, for compounds of formula VIII,

R^{x} is phenyl, 3-pyridyl or 3-fluoro-phenyl;

R^{w} is CH₂CH(Me)₂, cyclohexyl-CH₂—, para-hydroxybenzyl, CH₂C(Me)₃, C(Me)₃, cyclopentyl or cyclohexyl;

R^{z} is phenyl or thienyl, each of which may be optionally substituted by one or more substituents selected from OH, halo, alkyl, alkyl-NH₂, N-piperazinyl and N-piperidinyl, wherein said N-piperazinyl and N-piperidinyl are each optionally substituted by one or more alkyl or acyl groups. Additionally, R^{z} may be 2-furanyl, 3-furanyl or N-morpholinyl, each of which may be optionally substituted by one or more alkyl groups.

Preferably, for compounds of formula VIII,

R^{x} is phenyl;

R^{w} is CH₂CH(Me)₂, cyclohexyl-CH₂—, para-hydroxybenzyl, CH₂C(Me)₃ or C(Me)₃;

R^{z} is phenyl or thienyl each of which may be optionally substituted by one or more substituents selected from OH, halo, alkyl, alkyl-NH₂, N-piperazinyl and N-piperidinyl, wherein said N-piperazinyl and N-piperidinyl are each optionally substituted by one or more alkyl or acyl groups.

Further details of how to modify the compounds of formula I to form compounds of formula VII and VIII may be found in Quibell, M. et al, Bioorg. Med. Chem. 13 (2005), 609-625.

In one particularly preferred embodiment, said compound of formula I is converted to a compound of formula VIII by the steps set forth in Scheme I below. Firstly, said compound of formula I is coupled with a compound of formula R^{z}CONHCHR^{w}COOH (for example, using an acid activation technique) to form a compound of formula X. Said compound of formula X is then treated with a reagent capable of removing the R¹ group (for example, by acidolysis), and subsequently coupled with a carboxylic acid of formula R^{x}COOH to form a compound of formula XI. Said compound of formula XI is subsequently oxidised to form a compound of formula VIII.

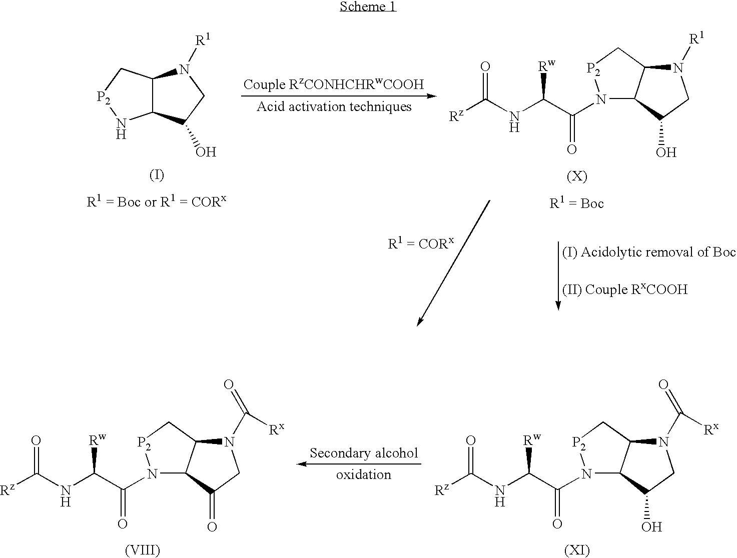

Suitable agents for the secondary alcohol oxidation step will be familiar to the skilled artisan. By way of example, the oxidation may be carried out via a Dess-Martin periodinane reaction [Dess, D. B. et al, J. Org. Chem. 1983, 48, 4155; Dess, D. B. et al, J. Am. Chem. Soc. 1991, 113, 7277], or via a Swern oxidation [Mancuso, A. J. et al, J. Org. Chem. 1978, 43, 2480]. Alternatively, the oxidation can be carried out using SO₃/pyridine/Et₃N/DMSO [Parith, J. R. et al, J. Am. Chem. Soc. 1967, 5505; U.S. Pat. No. 3,444,216, Parith, J. R et al,], P₂Os/DMSO or P₂O₅/Ac₂O [Christensen, S. M. et al, Organic Process Research and Development, 2004, 8, 777]. Other alternative oxidation reagents include activated dimethyl sulphoxide [Mancuso, A. J., Swern, D. J., Synthesis, 1981, 165], pyridinium chlorochromate [Pianeatelli, G. et al, Sythesis, 1982, 2451 and Jones' reagent [Vogel, A, I., Textbook of Organic Chemistry, 6^{th} Edition].

In another particularly preferred embodiment, the invention relates to a method of preparing a cysteinyl proteinase inhibitor of formula IX

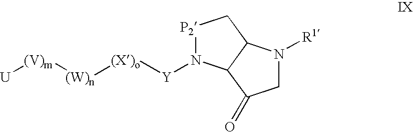

wherein:

P_{2′}═O, CH₂ or NR⁹, where R⁹ is chosen from H, C₁₋₇-alkyl, C₃₋₆-cycloalkyl, Ar or Ar—C₁₋₇-alkyl;

Y═CR¹⁰R¹¹—C(O) or CR¹⁰R¹¹—C(S) or CR¹⁰R¹¹—S(O) or CR¹⁰R¹¹—SO₂ where R¹⁰ and R¹¹ are independently chosen from H, C₁₋₇-alkyl, C₃₋₆-cycloalkyl, Ar and Ar—C₁₋₇-alkyl, or Y represents

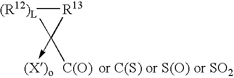

where L is a number from one to four and R¹² and R¹³ are independently chosen from CR¹⁴R¹⁵ where R¹⁴ and R¹⁵ are independently chosen from H, C₁₋₇-alkyl, C₃₋₆-cycloalkyl, Ar, Ar—C₁₋₇-alkyl or halogen; and for each R¹² and R¹³ either R¹⁴ or R¹⁵ (but not both R¹⁴ and R¹⁵) may additionally be chosen from OH, O—C₁₋₇-alkyl, O—C₃₋₆-cycloalkyl, OAr, O—Ar—C₁₋₇-alkyl, SH, S—C₁₋₇-alkyl, S—C₃₋₆-cycloalkyl, SAr, S—Ar—C₁₋₇-alkyl, NH₂, NH—C₁₋₇-alkyl, NH—C₃₋₆-cycloalkyl, NH—Ar, NH—Ar—C₁₋₇-alkyl, N—(C₁₋₇-alkyl)₂, N—(C₃₋₆-cycloalkyl)₂, NAr₂ and N-(Ar—C₁₋₇-alkyl)₂;
in the group (X′)ₒ, X′═CR¹⁶R¹⁷, where R¹⁶ and R¹⁷ are independently chosen from H, C₁₋₇-alkyl, C₃₋₆-cycloalkyl, Ar and Ar—C₁₋₇-alkyl and o is a number from zero to three;
in the group (W)ₙ, W═O, S, C(O), S(O) or S(O)₂ or NR¹⁸, where R¹⁸ is chosen from H, C₁₋₇-alkyl, C₃₋₆-cycloalkyl, Ar and Ar—C₁₋₇-alkyl and n is zero or one;
in the group (V)ₘ, V═C(O), C(S), S(O), S(O)₂, S(O)₂NH, OC(O), NHC(O), NHS(O), NHS(O)₂, OC(O)NH, C(O)NH or CR¹⁹R²⁰, C═N—C(O)—OR¹⁹ or C═N—C(O)—NHR¹⁹, where R¹⁹ and R²⁰ are independently chosen from H, C₁₋₇-alkyl, C₃₋₆-cycloalkyl, Ar, Ar—C₁₋₇-alkyl and m is a number from zero to three, provided that when m is greater than one, (V)ₘ contains a maximum of one carbonyl or sulphonyl group;

U=a stable 5- to 7-membered monocyclic or a stable 8- to 11-membered bicyclic ring which is saturated or unsaturated and which includes zero to four heteroatoms, selected from the following:

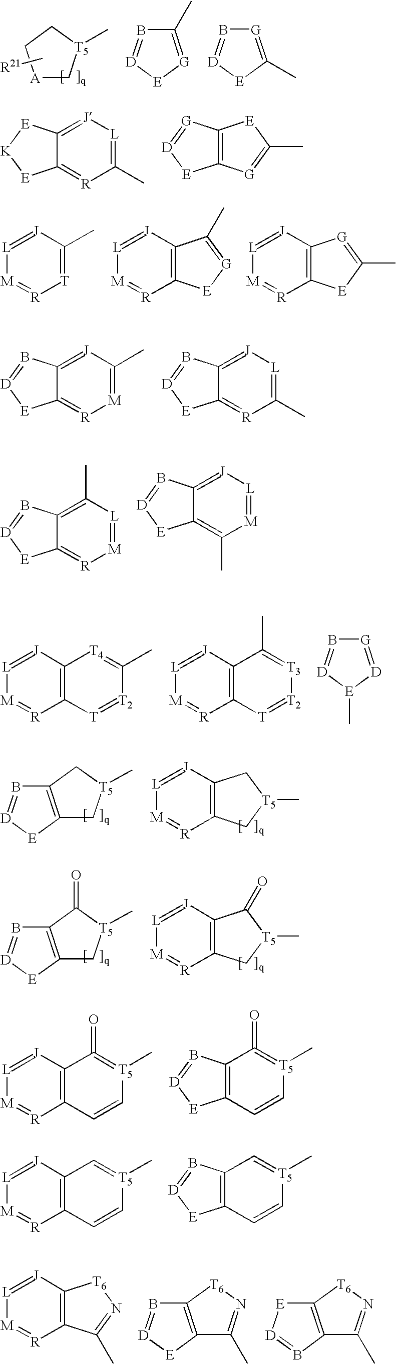

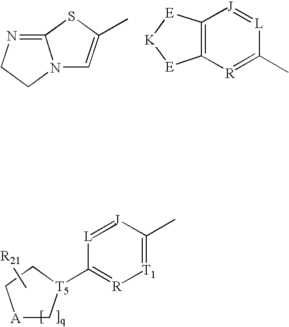

wherein R²¹ is:
H, C₁₋₇-alkyl, C₃₋₆-cycloalkyl, Ar, Ar—C₁₋₇-alkyl, OH, O—C₁₋₇-alkyl, O—C₃₋₆-cycloalkyl, O—Ar, O—Ar—C₁₋₇-alkyl, SH, S—C₁₋₇-alkyl, S—C₃₋₄-cycloalkyl, S—Ar, S—Ar—C₁₋₇-alkyl, SO₂H, SO₂—C₁₋₇-alkyl, SO₂—C₃₋₆-cycloalkyl, SO₂—Ar, SO₂—Ar—C₁₋₇-alkyl, NH₂, NH—C₁₋₇-alkyl, NH—C₃₋₆-cycloalkyl, NH—Ar, N—Ar₂, NH—Ar—C₁₋₇-alkyl, N(C₁₋₇-alkyl)₂, N(C₃₋₆-cycloalkyl) or N(Ar—C₁₋₇-alkyl)₂; or, when part of a CR²¹ or CR²¹ group, R²¹ may be halogen;
   A is chosen from:
CH₂, CHR²¹, O, S, SO₂, NR¹¹ or N-oxide (N→O), where R²¹ is as defined above; and R²² is chosen from H, C₁₋₇-alkyl, C₃₋₆-cycloalkyl, Ar and Ar—C₁₋₇-alkyl;
   B, D and G are independently chosen from:
CR²¹, where R²¹ is as defined above, or N or N-oxide (N→O);
   E is chosen from:
CH₂ CHR²¹, O, S, SO₂, NR²² or N-oxide (N—O), where R²¹ and R²² are defined as above;
   K is chosen from:
CH₂, CHR²², where R²² is defined as above;
   J, L, M, R, T, T₂, T₃ and T₄ are independently chosen from:
CR²¹ where R²¹ is as defined above, or N or N-oxide (N→O);
   T₅ is chosen from:
CH or N;
   T₆ is chosen from:
NR²², SO₂, OC(O), C(O), NR²²C(O);
   q is a number from one to three, thereby defining a 5-, 6- or 7-membered ring;
   R^{1′}═R^{2′}C(O), R^{2′}OC(O), R^{2′}OC(O), R^{2′}SO₂, where R² is chosen from C₁₋₇-alkyl, C₃₋₆-cycloalkyl, Ar and Ar—C₁₋₇-alkyl and Q is H or C₁₋₇-alkyl.

Further details of how to modify compounds of formula I to form compounds of formula IX may be found in WO 04/007501 (Amura Therapeutics Limited).

A further aspect of the invention relates to a method for preparing compounds of formula VII, VIII or IX as defined above, said method comprising the use of a process as defined above for said first aspect.

The present invention is further described by way of the following non-limiting examples.

### EXAMPLES

A highly preferred embodiment of the invention is set forth below in Scheme 2.

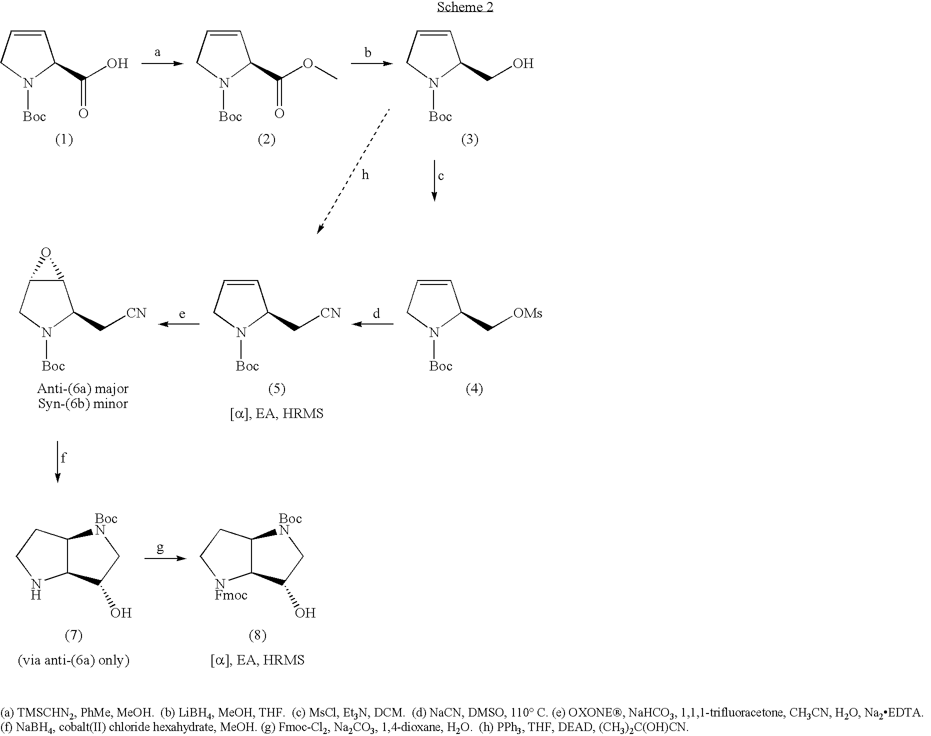

### Preparation of (S)-2,5-dihydropyrrole-1,2-dicarboxylic acid 1-tert-butyl ester 2-methyl ester (2)

(Trimethylsilyl)diazomethane (2.0 M solution in hexane, 200 mL, 400 mmol) was added dropwise over 15 minutes to a stirred mixture of toluene (600 mL), methanol (100 mL) and (S)-Boc-3,4-dehydroproline (1) (ex. Bachem, 50 g, 234.4 mmol) whilst cooling with iced-water under an atmosphere of argon. The yellow solution was stirred for 30 minutes then acetic acid 15 mL was added to obtain a colourless solution. The solvents were removed in vacuo to leave ester (2) (56.58 g, >100% yield) as a pale yellow oil which was used without further purification. TLC (single UV spot, R_{f}=0.10, heptane: ethyl acetate 1:1); analytical HPLC single main peak, Rₜ=14.26 min., HPLC-MS 128.2 [M+2H-Boc]⁺, 172.1 [M+2H-Bu]⁺, 477.3 [2M+Na]⁺.

### Preparation of (S)-2-hydroxymethyl-2,5-dihydropyrrole-1-carboxylic acid tert-butyl ester (3)

Lithium borohydride (10.21 g, 469.0 mmol) was suspended in THF (1000 mL), then methanol (19.3 mL) followed by a solution of ester (2) (53.3 g, 234.5 mmol) in dry THF (1428 mL) were added dropwise. After addition, the mixture was stirred for 1 hour at ambient temperature then water (608 mL) was cautiously added to the mixture, followed by extraction with dichloromethane (3×2026 mL). The combined organic layers were dried (MgSO₄). The filtrate was evaporated under reduced pressure to afford alcohol (3) (46.4 g, 99%) as a pale yellow oil which was used without further purification. TLC (R_{f}=0.20, heptane: ethyl acetate 1:1), analytical HPLC single main peak, Rₜ=11.32 min., HPLC-MS 100.2 [M+2H-Boc]⁺, 144.1 [M+2H-Bu]⁺, 222.0 [M+Na]⁺, 421.3 [2M+Na]⁺.

### Preparation of (S)-2-methanesulfonyloxymethyl-2,5-dihydropyrrole-1-carboxylic acid tert-butyl ester (4)

Triethylamine (52.3 mL, 372.4 mmol) was added dropwise to a stirred solution of alcohol (3) (46.4 g, 232.8 mmol) and methanesulfonyl chloride (27.0 mL, 349.2 mmol) in dichloromethane (200 mL) at 0° C. The mixture was stirred for 30 minutes at ambient temperature then washed with water (400 mL) and brine (400 mL). The organic layer was dried (Na₂SO₄), and concentrated in vacuo to obtain a pale yellow oil (65.2 g) which was purified by flash chromatography over silica, eluting with ethyl acetate:heptane mixtures to give mesylate (4) (57.9 g, 90%) as a pale yellow oil. TLC (R_{f}=0.15, heptane: ethyl acetate 1:1), analytical HPLC single main peak, Rₜ=10.21 min., HPLC-MS 178.1 [M+2H-Boc]⁺, 222.1 [M+2H-Bu]⁺, 300.1 [M+Na]⁺, 577.2 [2M+Na]⁺.

### Preparation of (S)-2-cyanomethyl-2,5-dihydropyrrole-1-carboxylic acid tert-butyl ester (5)

Sodium cyanide (30.7 g, 626.5 mmol) was added to a stirred solution of mesylate (4) (57.9 g, 208.8 mmol) in DMSO (400 mL) at ambient temperature. The mixture was heated at 110° C. for 1 hour before being allowed to cool to ambient temperature then poured into dichloromethane (400 mL) and water (400 mL). The organic layer was separated then the aqueous was extracted with dichloromethane (3×100 mL). The combined dichloromethane layers were washed with brine (200 mL), dried (MgSO₄), and evaporated in vacuo to leave a residue which was purified by flash chromatography over silica, eluting with ethyl acetate: heptane mixtures to give nitrile (5) as an oil which solidified to a white waxy solid upon refrigeration (37.6 g, 87%). TLC (R_{f}=0.40, heptane: ethyl acetate 1:1), analytical HPLC single main peak, Rₜ=14.77 min., HPLC-MS 153.2 [M+2H-Bu]⁺, 209.2 [M+1]⁺, 231.1 [M+Na]⁺, 439.3 [2M+Na]⁺. _{δ}H (CDCl₃ at 298K); mixture of rotamers 1.39-1.55 (9H, two s, C(CH₃)₃), 2.70-2.78 and 3.00-3.10 (2H, m, CHCH₂CN), 4.08-4.20 (2H, m, CH₂N CO₂), 4.62-4.78 (1H, m, CHNCO₂), 5.70-5.80 and 5.93-6.07 (2H, CH═CH). _{δ}C(CDCl₃ at 298K); 22.51, 23.58 (CH₂CN), 29.66 (C(CH₃)₃), 53.83, 54.00 (CH₂N CO₂), 60.43, 60.53 CHNCO₂), 80.35, 80.74 (C(CH₃)₃), 116.86, 117.17 (CN), 126.86, 126.92 (CH═CH), 128.77, 128.85 (CH═CH), 153.44, 153.98 (C═O); [α]_{D}²²−290.7° (c 0.269, CHCl₃); Anal. calcd for C₁₁H₁₆N₂O₂: C, 63.44; H, 7.74; N, 13.45; found C, 63.23; H, 7.63; N, 13.31; Exact mass calcd for C₁₁H₁₆N₂O₂ (MNa⁺): 231.1104, found 231.1096 (−3.22 ppm).

### Alternative Preparation of (S)-2-cyanomethyl-2,5-dihydropyrrole-1-carboxylic acid tert-butyl ester (5)

To a solution of alcohol (3) (0.204 g, 1.024 mmol) in THF (10 mL) at 0° C. was added triphenylphosphine (0.537 g, 2.048 mmol). The reaction mixture was stirred at 0° C. (ice-water bath) for 10 minutes. Then DEAD (0.357 g, 2.048 mmol) was added dropwise and the mixture was stirred for 20 minutes. Acetone-cyanohydrin (0.174 g, 2.048 mmol) was added dropwise. After the addition, the mixture was allowed to warm to room temperature under stirring for 26 hours. The solvent was removed under reduced pressure to afford the crude product. The crude product was purified by Jones ISOLUTE Flash-XL Si II then I·(20 g) X 2 column chromatography using n-heptane: ethylacetate=8:1 to 6:1 to give product as an off-white oil (0.134 g, 63%). TLC (Rf=0.4, n-heptane:ethylacetate 1:1)., HPLC-MS (V peak with Rt=4.080, 153.2 [M+1-56]⁺, 209.2 [M+1]⁺, 231.1 [M+Na]⁺, 439.3 [2M+Na]⁺._{δ}H(CDCl₃ at 298K); 1.39-1.55 (9H, C(CH₃, bd), 2.70-2.78, 3.00-3.10 (2H, NCCH₂, m), 4.08-4.20 (2H, CHCH₂N, m), 4.62-4.78 (1H, CHCHCH₂N, m), 5.70-5.80, 5.93-6.07 (2H, CH═CH, m)._{δ}C(CDCl₃ at 298K); 22.51, 23.58 (d, NCCH₂), 29.66 (u, CH₃), 53.83, 54.00 (d, CHCH₂N), 60.43, 60.53 (u, NCHCH₂CN), 80.35, 80.74 (q, C(CH₃)₃), 116.86, 117.17 (q, CN), 126.86, 126.92 (u, CH═CH), 128.77, 128.85 (u, CH═CH), 153.44, 153.98 (q, CO).

### Preparation of (2R,3R,4S)-2-cyanomethyl-6-oxa-3-azabicyclo[3.1.0]hexane-3-carboxylic acid tert-butyl ester (6a)

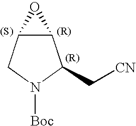

To a solution of nitrile (5) (6 g, 28.85 mmol) in acetonitrile (150 mL) and aqueous Na₂.EDTA (150 mL, 0.4 mmol solution) at 0° C. was added 1,1,1-trifluoroacetone (31.0 mL, 346 mmol) via a pre-cooled syringe. To this homogeneous solution was added in portions a mixture of sodium bicarbonate (20.4 g, 248 mmol) and OXONE® (55.0 g, 89.4 mmol) over a period of 1 hour. The mixture was then diluted with water (750 mL) and the product extracted into dichloromethane (4×150 mL). The combined organic layers were washed with 5% aqueous sodium hydrogen sulfite (300 mL), water (300 mL) and brine (300 mL) then dried Na₂SO₄, and evaporated in vacuo to leave a residue which was recrystallised from diethyl ether:heptane (1:6) to give (3R,4S)-2R-cyanomethyl-6-oxa-3-azabicyclo[3.1.0]hexane-3-carboxylic acid tert-butyl ester (6a) as a white solid (4.3 g, 67%). TLC(R_{f}=0.20, n-heptane: ethyl acetate 1:1), HPLC-MS 169.1 [M+2H-Bu]⁺, 247.1 [M+Na]⁺, 471.3 [2M+Na]⁺._{δ}H(CDCl₃ at 298K); 1.43-1.47 (9H, two s, (CH₃)₃C), 2.60-3.02 (2H, CHCH₂CN, m), 3.46-3.65 (2H, CHOCH, m), 3.75-3.92 (2H, CH₂NCO₂, m), 4.17-4.24 (1H, CHNCO₂, m). _{δ}C(CDCl₃ at 298K); 19.07, 19.94 (CHCH₂CN), 28.31, 28.37 (C(CH₃)₃), 46.82, 47.56 CH₂NCO₂), 54.14, 54.38 CHNCO₂), 54.70, 55.54 (CHOCH), 57.32, 57.78 (CHOCH), 80.91, 81.18 (C(CH₃)₃), 116.46, 116.95 (CN), 153.74, 154.27 (CO); [α]_{D}²²-159.2° (c 0.628, CHCl₃). An additional crop of product was obtained as a 6:1 mixture of (3R,4S)-2R-cyanomethyl-6-oxa-3-azabicyclo[3.1.0]hexane-3-carboxylic acid tert-butyl ester (6a): (3S,4R)-2R-cyanomethyl-6-oxa-3-azabicyclo[3.1.0]hexane-3-carboxylic acid tert-butyl ester (6b) following flash chromatography then recrystallisation of the mother liquors (444 mg, 7%).

### Preparation of (3aS,6aR)-3S-hydroxyhexahydropyrrolo[3,2-b]pyrrole-1-carboxylic acid tert-butyl ester (7)

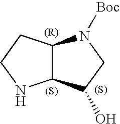

Sodium borohydride (0.42 g, 11.20 mmol) was added in portions over 30 minutes to a solution of cobalt(n) chloride hexahydrate (0.53 g, 2.23 mmol) and epoxide (6a) and (0.5 g, 2.23 mmol) in methanol (20 mL) at 0° C. After the addition, the mixture was left to stir at ambient temperature for 1 hour then citric acid (25 mL, 10% aqueous solution) was added dropwise over 10 minutes (pH ˜4). Sodium hydroxide (5M) was then added whilst cooling with iced-water until pH ≧13 was reached, then the mixture was extracted with dichloromethane (10×20 mL), dried (Na₂SO₄), and evaporated in vacuo to give (3aS,6aR)-3S-hydroxyhexahydropyrrolo[3,2-b]pyrrole-1-carboxylic acid tert-butyl ester (7) (0.41 g, 80%) as a colourless oil which was used without further purification. HPLC-MS UV peak 173.1 [M+2H-Bu]⁺, 229.1 [M+1]⁺, 251.1 [M+Na]⁺. δ_{H} (400 MHz, CDCl₃) approximately 1:1 mixture of rotamers 1.55 (9H, s, C(CH₃)₃), 1.92 and 2.03 (2H total, each br. s, NHCH₂CH₂), 2.71 and 2.79 (2H total, m, NHCH₂CH₂), 3.46 (1H, dd, J=12.15 and 3.80 Hz, BocNCH₂), 3.74-3.62 (1H, m, BocNCH₂), 3.60-3.69 (1H, m, CHNHCH₂), 4.10 (1H, s, CHOH), 4.33 and 4.40 (1H total, each s, BocNCHCH₂).

### Preparation of (3aS,6aR)-3S-hydroxyhexahydropyrrolo[3,2-b]pyrrole-1,4 dicarboxylic acid 1-tert-butyl ester 4-(9H-fluoren-9-ylmethyl) ester (8)

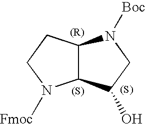

A solution of 9-fluorenylmethyl chloroformate (0.130 g, 0.504 mmol) in 1,4-dioxane (3 mL) was added dropwise over 40 min whilst stirring to a solution of (3aS,6aR)-3S-hydroxyhexahydropyrrolo[3,2-b]pyrrole-1-carboxylic acid tert-butyl ester (7) (0.1 g, 0.438 mmol) and sodium carbonate (0.104 g, 0.986 mmol) in water (2 mL) and 1,4-dioxane (3 mL) at 0° C. After the addition, the mixture was stirred at ambient temperature for 1 hour then water (50 mL) added and mixture extracted with dichloromethane (4×50 mL), dried (Na₂SO₄), and evaporated in vacuo to leave a residue which was purified by flash chromatography over silica, eluting with ethyl acetate:heptane mixtures to give (3aS,6aR)-3S-hydroxyhexahydropyrrolo[3,2-b]pyrrole-1,4-dicarboxylic acid 1-tert-butyl ester 4-(9H-fluoren-9-ylmethyl) ester (8) (0.152 g, 77%) as an off-white solid. HPLC peak with Rₜ=18.582 min., HPLC-MS 351.2 [M+2H-Boc]⁺, 395.2 [M+2H-Bu]⁺, 451.3 [M+H]⁺, 473.2 [M+Na]⁺, 923.5 [2M+Na]⁺. _{δ}H (CDCl₃ at 298K); mixture of rotamers, 1.33-1.52 (9H, two s, C(CH₃)₃), 1.58-1.75 and 1.90-2.21 (4H, m, CH₂CH₂), 2.85-3.66 (5H, m, NCH₂CHOH and NCHCH, 4.02-4.83 (3H, m, FmocCH and CH₂), 7.25-7.83 (8H, Fmoc aromatic). ₆₇C (CDCl₃ at 298K); 29.28 (C(CH₃)₃), 33.06, 33.23 (CH₂CH₂NFmoc), 46.35, 46.60 (CH₂CH₂NFmoc), 48.93 (Fmoc-CH), 54.73, 55.34 (CH₂NBoc), 61.83, 62.84 (CHNBoc), 68.05, 68.26 (Fmoc-CH₂), 68.88, 69.49, 69.69, 70.27 (CHNFmoc), 73.06, 73.61, 73.94, 74.57 (CHOH), 80.63 (C(CH₃)₃), 121.59, 126.75, 128.74, 129.33 (Fmoc CH aromatics), 142.85, 145.72, 145.91 (Fmoc quaternary aromatics), 155.41, 155.59, 155.82 (NCO₂).; [α]_{D}²²-1020.0° (c 0.457, CHCl₃); Anal. calcd for C₂₆H₃₀N₂O₅: C, 69.31; H, 6.71; N, 6.22; found C, 69.11; H, 7.06; N, 5.84; Exact mass calcd for C₂₆H₃₀N₂O₅ (MNa⁺): 473.2052, found 473.2053 (+0.06 ppm).

### Variation in Cyclisation Routes

An alternative order of reactions towards bicycle (7) has been investigated and is detailed in Scheme 3.

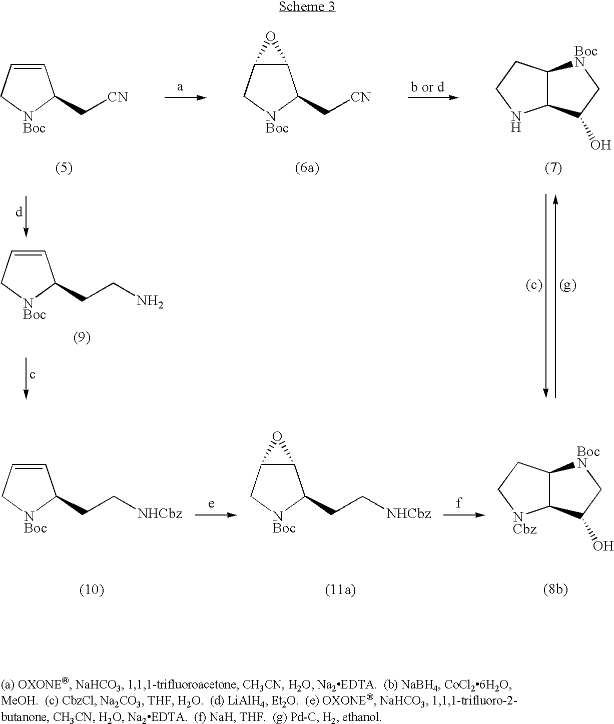

Useful bicyclic derivatives such as the Boc-Cbz alcohol (8b) can be prepared from nitrile (5) by a variety of routes (see Scheme 3). However, a comparison of the routes shown suggests that the preferred choice is that outlined in Scheme 2 which utilises the crystallisation of (6a) as a key advantage. Thus, using the reaction sequence of epoxidation then nitrile reduction with cobalt catalysis (a→b→c) an overall yield of 68% can be achieved for the synthesis of (8b), which may be quantitatively hydrogenated to bicycle (7). In comparison, two alternative sequences comprising epoxidation then nitrile reduction with lithium aluminium hydride (a→d→c) or nitrile reduction, amine protection, epoxidation, hydrogenation/intramolecular cyclisation (d→c→e→f) led to 39% and 22% overall yields respectively of (8b). Although conditions for the later route were not optimised (e.g. improved stereochemical control of epoxidation through OXONE®, NaHCO₃, 1,1,1-trifluoro-2-butanone, CH₃CN, H₂O, Na₂.EDTA and possible recrystallisation of (IIa)), the extra steps compared that for Scheme 2 appear to offer no advantage.

A more highly preferred embodiment of the invention is now set forth below in Scheme 4 that details optimum conditions for the reactions described in Scheme 2.

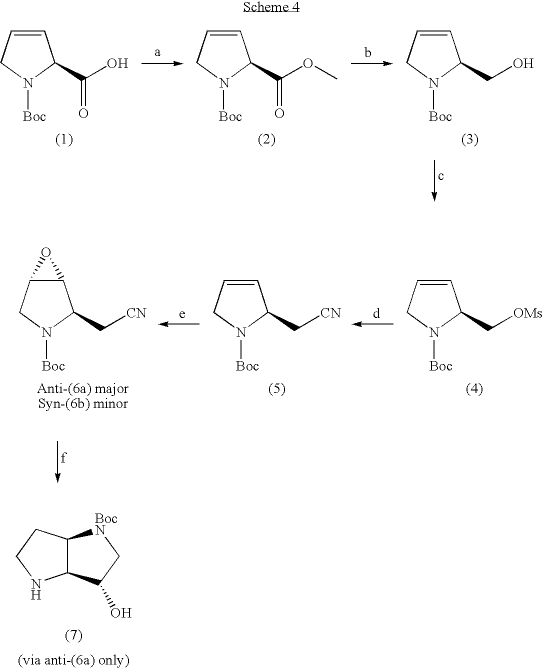

### Alternative Large Scale Preparation of (S)-2,5-dihydropyrrole-1,2-dicarboxylic acid 1-tert-butyl ester 2-methyl ester (2)

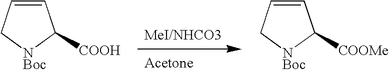

| | Weight | | | |
|---|---|---|---|---|
| Material | (gm) | Mol. weight | Moles | Mole equivalent |
| Acid (1) | 25.0 | 213.23 | 0.117 | 1 |
| Methyl Iodide | 50.0 | 141.94 | 0.352 | 3 |
| Acetone | 200 mls | / | / | 8 vol |
| KHCO₃ | 17.6 | 100.12 | 0.176 | 1.5 |
| MDC | 100 mls | / | / | 4 vol |

Stage a 250 ml 4 neck RBF fitted with over head stirrer, thermo-pocket and chilled water condenser.
Charge acid (1) (25.0 g) and acetone (175 mls) and stir to dissolve.
Charge KHCO3 (17.6 g) at 30-35° C. and flush the funnel with 25 ml of acetone.
Charge methyl iodide (50.0 g) slowly via a dropping funnel over the 15-20 min maintaining a temperature of 30-35° C.
Set the reaction for reflux (43-45° C.) and monitor the reaction by TLC System (toluene: Methanol; 9:1) until complete (about 5-6 hrs).
After completion of the reaction, cool the reaction mixture to 15-20° C. and filter through a celite bed.
Distill under vacuum at 40-45° C. until the reaction mixture becomes a thick suspension.
Charge MDC (75 mls) to the suspension to ensure the product remains in solution.
Filter the slurry through a celite bed and wash the cake with 25 ml MDC.
Concentrate the filtrate under vacuum at 45-50° C. to give the product as a light yellow liquid.

| Weight of product | 26.4 g (99.2%) |
|---|---|
| Purity By GC | 99.8% |

[product identity confirmed by ¹H, ¹³C nmr]

### Alternative Large Scale Preparation of (S)-2-hydroxymethyl-2,5-dihydropyrrole-1-carboxylic acid tert-butyl ester (3)

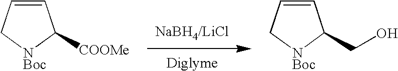

| | Weight | | | |
|---|---|---|---|---|
| Material | (gm) | Mol. weight | Moles | Mole equivalent |
| Ester (2) | 25.0 | 227.26 | 0.11 | 1.0 |
| LiCl | 4.7 | 42.34 | 0.11 | 1.0 |
| NaBH4 | 4.2 | 37.8 | 0.11 | 1.0 |
| Diglyme | 50 ml | / | / | 2 vols |
| 1N HCL | 190 ml | / | / | — |
| Toluene | 750 ml | / | / | 30 vols |
| Pure water | 500 ml | / | / | 10 vol w.r.t |
| | | | | diglyme |

Stage a 500 ml 4 neck RBF fitted with overhead stirrer and condenser.
Charge ester (2) (25.0 g) and diglyme (25 mls) and stir at 30-35° C. and stir to dissolve.
Charge LiCl (4.7 g) and NaBH4 (4.2 g) in one lot and flush the funnel with 25 ml warm diglyme.
Stir the reaction mixture for 15 min at 30-35° C. and then increase the temperature to 90-95° C. Maintain this temperature until the reaction is complete. (90-100 minutes). The reaction is monitored by TLC (ethyl acetate:Hexane; 4:6).
After completion of reaction, cool the reaction mass to RT. Add 500 ml DI water slowly to the above mass (exothermic and effervescent!) over 30 minutes.
Adjust the pH of the reaction mixture to ˜4 using 1N HCl.
Add toluene (250 ml) to the reaction mixture and stir for 10-15 min at 30-35° C. and separate the layers.
Repeat the toluene extraction twice (2×250 mls).
Combine organic layer and wash with water (1×250 mls). Note wash was stirred for 15 minutes prior to settling the layers.
The organic layer is dried over MgSO₄ and concentrated under vacuum at 50-55° C. to give the product as an oil.

| Weight of the Liquid | 22.4 g (>100%, contains some solvent) |
|---|---|
| Purity By GC | 98% |

[Product structure confirmed by ¹H, ¹³C nmr]

### Alternative Large Scale Preparation of (S)-2-methanesulfonyloxymethyl-2,5-dihydropyrrole-1-carboxylic acid tert-butyl ester (4)

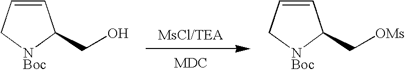

| | Weight | Mol. | | |
|---|---|---|---|---|
| Material | (gm) | weight | Moles | Mole equivalent |
| Alcohol (3) | 20.0 | 199.25 | 0.100 | 1.0 |
| CH₃SO₂Cl | 17.3 | 114.55 | 0.1506 | 1.5 |
| TEA | 20.2 | 101.10 | 0.200 | 2.0 |
| MDC | 80 mls | — | — | 4 vols |
| DI water | 160 mls | — | — | 2 vol w.r.t mdc |
| 20% NaCl solution | 160 mls | — | — | 2 vol w.r.t mdc |
| DI water | 80 mls | — | — | |

Stage a 500 ml 4 Neck RBF fitted with overhead stirrer and ice bath.
Charge alcohol (3) (20.0 g) and MDC (80 mls) and stir to dissolve.
Charge methanesulfonyl chloride (17.3 g) slowly to the reaction mixture over 10-15 minutes maintaining a reaction temperature of 30-35° C.
Stir the mass for 10 min at 30° C. and cool to 0-2° C.
Charge TEA (20.2 g) slowly to (highly exothermic reaction) maintaining the temperature at 0-8° C.
Allow the reaction to warm to room temperature and stir until the reaction is complete (90-100 min), monitoring by TLC (Toluene:methanol; 9:1).
Charge DI water (160 mls) to the reaction mixture and stir for 10-15 min at 28-30° C.
Separate layers and extract the aqueous layer with MDC (3×50 mls) stirring for 15 minutes between extractions.
Wash the organic layer with 0.1N HCl (1×50 mls), say NaHCO₃ solution (50 mls) and brine (160 mls).
dry the organic layer over MgSO₄ and strip the solvent under vacuum (40-50° C.) to give the product as a viscous oil.

| | |
|---|---|
| Weight of product | 22.3 g (80%) |

Product identity confirmed by 1H NMR

### Alternative Large Scale Preparation of (S)-2-cyanomethyl-2,5-dihydropyrrole-1-carboxylic acid tert-butyl ester (5)

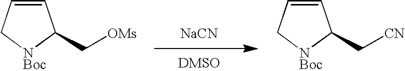

| | Weight | Mol. | | |
|---|---|---|---|---|
| Material | (gm) | weight | Moles | Mole equivalent |
| Mesylate (4) | 20.0 | 277.34 | 0.0722 | 1 |
| NaCN | 5.3 | 49.0 | 0.108 | 1.5 |
| DMSO | 100 ml | — | — | 5 volumes |
| Toluene | 600 ml | | | |
| DI water | 1000 ml | — | — | 10 vol w.r.t dmso |
| | +100 | | | |

Stage a 500 ml 4 neck RBF fitted with overhead stirrer and water condenser.
Charge 20.0 g of mesylate (4) and 80 mls DMSO and stir for 5 min at 30° C. to dissolve.
Charge 5.3 g NaCN in one lot and flush the funnel with DMSO (20 mls) at 30° C. (vent reaction to a circulating bleach scrubber!)
a Heat the reaction mixture to 90-95° C. and maintain with stirring until the reaction is complete (˜2 hrs). Monitor the reaction by TLC using toluene:methanol (9:1).
Allow the reaction mixture to cool to room temperature and charge 1000 ml of DI water slowly to form a uniform solution.
Charge toluene (200 ml) and stir for 10 min.
Separate the layers.
Repeat the extraction with toluene (2×200 mls).
Wash the combined toluene layers with water (2×100 mls).
Treat the combined aqueous liquors to remove traces of cyanide before disposal.
Dry the organic layer over MgSO₄.
Concentrate the organic layer under vacuum (50-55° C.) to afford the product as a thick dark brown liquid.

| Weight of the Liquid | 9.9 g (66%) |
|---|---|
| Purity By GC | 95% |

[confirmed the product by ¹H, ¹³C nmr]

Additional product can be extracted from the combined aqueous layer.

### Alternative Large Scale Preparation of (2R,3R,4S)-2-cyanomethyl-6-oxa-3-azabicyclo[3.1.0]hexane-3-carboxylic acid tert-butyl ester (6a)

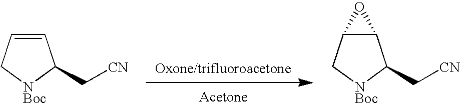

| | Weight | Mol. | | |
|---|---|---|---|---|
| Name | (gm) | weight | Moles | Mole equivalent |
| Cyanide (5) | 10.0 | 208.26 | 0.04807 | 1.0 |
| Oxone | 59.10 | 614.78 | 0.09614 | 2.0 |
| 1,1,1- | 10.7 | 112.05 | 0.09614 | 2.0 |
| trifluoroacetone | | | | |
| Acetone | 30.6 | 58 | 0.5287 | 11.0 |
| NaHCO3 | 34.72 | 84 | 0.413 | 8.6 |
| ACN | 200 ml | — | — | 20 vols |
| Na₂EDTA | 0.25 | 372.24 | 0.00067 | 0.014 |
| DI water | 1400 + | | | |
| | 250 ml | | | |
| MDC | 600 ml | | | |
| 5% NaSO₃ sol | 400 ml | | | |
| 20% NaCl sol | 400 ml | | | |

Stage a 500 ml 4 neck RBF fitted with overhead stirrer.
Charge cyanide (5) (10.0 g), ACN (200 mls) and stir the mixture for 5 min at RT.
Charge sodium EDTA solution (0.25 gr in 250 ml water).
Cool the reaction mixture to 0-5° C.
Charge 1,1,1-trifluoroacetone (10.7 g) directly into the 35 mls of pre cooled acetone (0-5° C.) and immediately add in one lot to the reaction mass (1,1,1-TFA is a highly volatile reagent!)
Add an intimate mixture of Oxone (59.1 g) and sodium bi carbonate (34.7 g) to the reaction mixture slowly over a period of 60-90 min at 0-5° C.
After the addition is complete, monitor by TLC using (6:4) Hexane:EtOAc.
Charge water (1 L) to the reaction mixture and stir to give a clear solution.
Charge MDC (200 mls) stir for 10-15 nm in at 20-25° C.
Separate the layers.
Repeat the extraction using MDC (2×200 mls).
Combined organic layer and wash with 5% aq sodium sulfite (400 mls), stirring the solution for 10 min.
Separate layers.
Charge water (400 mls) to the organic layer and stir for 10-15 min.
Separate layers.
Wash the organic layer with 20% NaCl solution (400 mls), stirring for 10-15 min prior to separation of layers.
Dry the organic layer over MgSO₄.
Concentrate under vacuum (45-50° C.) to give crude epoxide (10.2 grams) as a light yellow liquid.

| Weight of the Liquid | 10.2 gr (95%) |
|---|---|
| Purity By GC | 73% |

### Purification of (2R,3R,4S)-2-cyanomethyl-6-oxa-3-azabicyclo[3.1.0]hexane-3-carboxylic acid tert-butyl ester (6a)

Dissolve crude epoxide (10.0 g) MDC (25 mls) and charge neutral alumina (50 g) to adsorb the product.

Strip to dryness on a rotary evaporator to give a fine powder.

### First Extraction (Cyclohexane)

Add cyclohexane (50 mls) to the alumina/product mixture and stir for 15 min at 30-35° C. and filter. Repeat cyclohexane wash (3×50 mls). Combine extracts.

### 8:2 Extraction

To the alumina cake, add a 50 ml mixture of cyclohexane: EtOAc/(8:2), stir for 15 min and filter. Repeat the same extraction five more times (6×50 ml) and combine extracts.

### 6:4 Extraction

Extract the alumina six times with a 50 ml mixture of cyclohexane:EtOAc (6:4) (6×50 ml)

Concentrate the fractions separately.

| | |
|---|---|
| Weight of the Liquid | 7.4 gr (F-1 = 1.4 gr; F-2 = 5.2 gr; F-3 = 0.8 gr) |
| Purity By GC | NLT 80% |

### Re-Crystallization of Purified Purification of (2R,3R,4S)-2-cyanomethyl-6-oxa-3-azabicyclo[3.1. 0]hexane-3-carboxylic acid tert-butyl ester (6a)

Charge 6.0 gr purified epoxide to a flask fitted with overhead stirrer.
Charge 60 ml of 9:1 toluene/cyclohexane.
Stir for 30 min at 30° C.
White crystals start to form
Cool to 1° C. and stir for 1 hour.
Filter the product and dry under vacuum at 35° C. overnight.

| | |
|---|---|
| Weight of the solid: | 4.5 gr (44% theory) |
| Purity By GC | NLT 98% |

[Structure confirmed by ¹H, ¹³C nmr]

### Alternative Large Scale Preparation of (3aS,6aR)-3S-hydroxyhexahydropyrrolo[3,2-b]pyrrole-1-carboxylic acid tert-butyl ester (7)

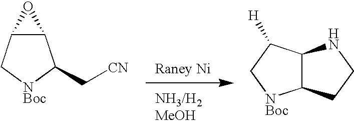

| | | Weight | Mol. | | Mole |
|---|---|---|---|---|---|
| No. | Name | (gm) | Wt | Moles | equiv |
| 1 | Anti-epoxide (6a) | 3.0 gr | 224.26 | 0.0133 | 1.0 |
| 2 | MeOH | 48 | | | 16.0 |
| | | mls | | | pts |
| 3 | Raney Ni | 5.0 gr | | | |
| 4 | 10% ammonia in MeOH | 50 ml | | | |

In a 1 lit autoclave (stirrer type) charged 3.0 gr anti-epoxide (6a) and 5.0 gr Raney Nickel and 48 mls methanol followed by 10% ammonia in methanol (50 mls) at 30-C.
4.0-4.5 kg Hydrogen pressure was maintained for 2.0 hrs
Reaction is monitored by TLC using 5% toluene in methanol
After completion of the reaction, material was filtered through hyflow and the filtrate was distilled off to get the final diamine as a thick liquid.
Yield 3.0 gr (98.2%)
[product identity confirmed by ¹H, ¹³C nmr]

In summary, the overall reaction sequence described in Scheme 2 to convert the carboxylic ester to bicyclic alcohol, via reduction, mesylation, cyanide displacement, epoxidation and reductive-cyclisation steps (Scheme 2), is clearly superior to the routes which use the reactions outlined in Scheme 3 (routes (d→c→e→f) or (a→d→c)). In particular, the low number of high yielding reactions, the use of (in general) non-chromatographic purification techniques, and the highly diastereoselective epoxide recrystallisation are all evidence that Scheme 2 is a superior process. The optimum conditions for the conversions detailed in Scheme 2 are detailed in Scheme 4.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

1. A process for preparing a compound of formula I, or a pharmaceutically acceptable salt thereof, 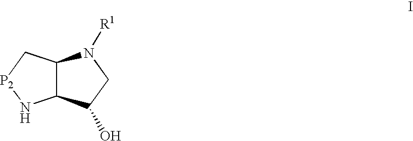
wherein
R¹ is Pg¹ or P₁′;
P₁′ is CO-hydrocarbyl;
P₂ is CH₂, O or N-Pg²; and
Pg¹ and Pg² are each independently nitrogen protecting groups;
said process comprising the steps of:
(i) reacting a compound of formula II with a dioxirane to form an epoxide of formula III; 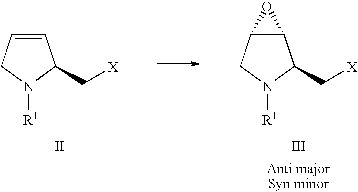
where X is selected from CN, CH₂N₃, CH₂NH-Pg², ONH-Pg², NHNH-Pg², N(Pg)NH-Pg²;
(ii) converting a compound of formula III to a compound of formula I

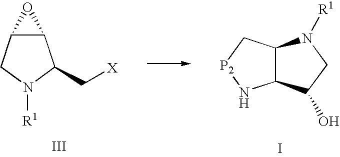

2. A process according to claim 1 wherein the dioxirane is generated in situ by the reaction of KHSO₅ with a ketone.

3. A process according to claim 1 or claim 2 wherein the ketone is of formula V 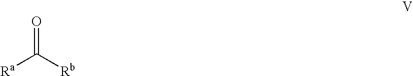
wherein R^{a} and R^{b} are each independently alkyl, aryl, haloalkyl or haloaryl.

4. A process according to claim 3 wherein R^{a} and R^{b} are each independently alkyl or haloalkyl.

5. A process according to claim 3 or claim 4 wherein R^{a} and R^{b} are each independently methyl or trifluoromethyl.

6. A process according to any one of claims 2 to 5 wherein the ketone is selected from acetone and a 1,1,1-trifluoroalkyl ketone.

7. A process according to claim 6 wherein the trifluoroalkyl ketone is 1,1,1-trifluoroacetone or 1,1,1-trifluoro-2-butanone.

8. A process according to any preceding claim wherein step (i) is carried out at a pH of from about 7.5 to about 8.

9. A process according to any preceding claim wherein step (i) is carried out in the presence of NaHCO₃.

10. A process according to any preceding claim wherein step (i) is carried out in a solvent comprising acetonitrile.

11. A process according to any preceding claim wherein step (i) is carried out in the in a solvent mixture which further comprises a phase transfer reagent.

12. A process according to any preceding claim wherein step (i) is carried out in the in a solvent mixture comprising aqueous Na₂.EDTA.

13. A process according to any preceding claim wherein step (ii) comprises converting a compound of formula III to a compound of formula IV in situ; and converting said compound of formula IV to a compound of formula I,

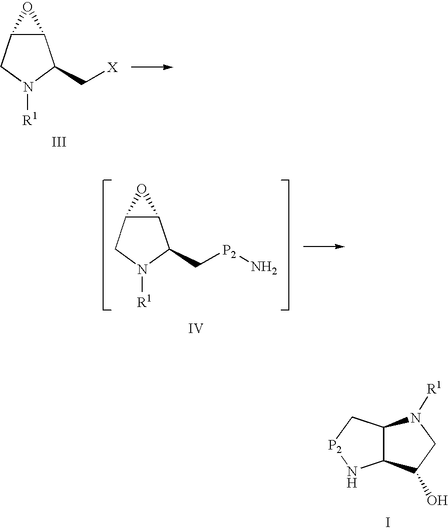

14. A process according to any preceding claim wherein X is CN.

15. A process according to any preceding claim wherein P₂ is CH₂.

16. A process according to any preceding claim wherein step (ii) comprises converting a compound of formula IIIa to a compound of formula IVa; and converting said compound of formula IVa to a compound of formula Ia

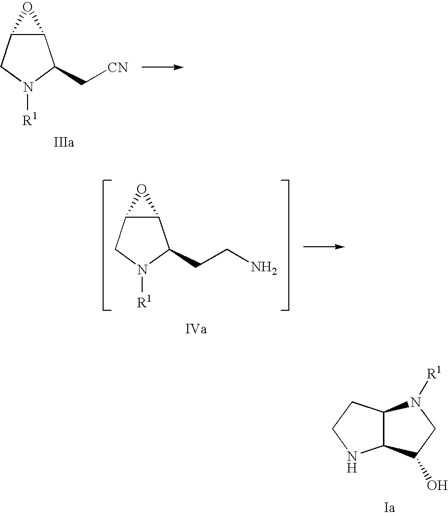

17. A process according to claim 16 wherein step (ii) comprises treating a compound of formula IIIa with sodium borohydride and cobalt (II) chloride hexahydrate.

18. A process according to claim 17 wherein the solvent for step (ii) is methanol.

19. A process according to claim 16 wherein R¹ is tert-butoxycarbonyl Boc and step (ii) comprises treating a compound of formula IIIa with Raney nickel and hydrogen.

20. A process according to claim 19 wherein the solvent for step (ii) is methanol containing ammonia.

21. A process according to any preceding claim wherein said compound of formula II is of formula IIa, 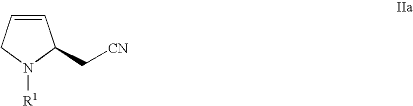
and R¹ is as defined in claim 1.

22. A process according to claim 21 wherein said compound of formula IIa is prepared from a compound of formula IIb, where LG is a leaving group 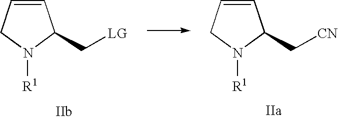
and R¹ is as defined in claim 1.

23. A process according to claim 22 wherein the leaving group, LG, is Ms, Ts, halo or OH.

24. A process according to claim 22 or claim 23 wherein said compound of formula IIa is prepared by reacting a compound of formula IIb with sodium cyanide.

25. A process according to claim 22 wherein the leaving group, LG, is Ms, and said compound of formula IIb is prepared by mesylating a compound of formula IIc 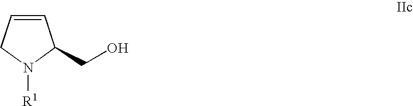
where R¹ is as defined in claim 1.

26. A process according to claim 22 wherein the leaving group, LG, is Ts, and said compound of formula IIb is prepared by tosylating a compound of formula IIc 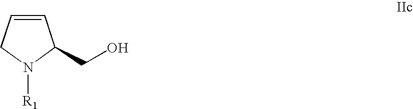
where R¹ is as defined in claim 1.

27. A process according to claim 22 wherein the leaving group, LG, is OH.

28. A process according to claim 27 wherein said compound of formula IIa is prepared by reacting a compound of formula IIc with triphenylphosphine, DEAD and acetone cyanohydrin 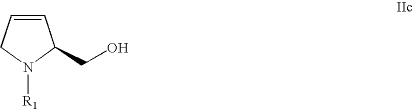
where R¹ is as defined in claim 1.

29. A process according to any one of claims 25 to 28 wherein said compound of formula IIc is prepared from a compound of formula IId 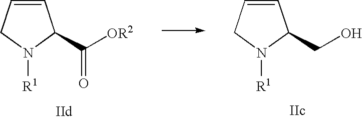
where R² is an alkyl or aryl group and R¹ is as defined in claim 1.

30. A process according to claim 29 wherein said compound of formula IIc is prepared by reacting a compound of formula IId with LiBH₄ in methanol/THF.

31. A process according to claim 29 wherein R¹ is tert-butoxycarbonyl (Boc) and said compound of formula IIc is prepared by reacting a compound of formula IId, wherein R² is methyl, with lithium chloride and sodium borohydride.

32. A process according to claim 31 which is carried out using diethylene glycol dimethyl ether (Diglyme) as solvent.

33. A process according to claim 29 wherein said compound of formula IId is prepared from a compound of formula IIe 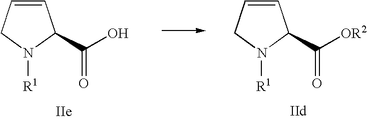
where R² is an alkyl or aryl group and R¹ is as defined in claim 1.

34. A process according to claim 33 wherein said compound of formula IId is prepared by reacting a compound of formula IIe with (trimethylsilyl)diazomethane in toluene/MeOH.

35. A process according to claim 33 wherein R¹ is tert-butoxycarbonyl (Boc) and said compound of formula IId, where R² is methyl, is prepared by reacting a compound of formula IIe with methyl iodide and potassium hydrogen carbonate.

36. A process according to claim 29 wherein said compound of formula IId is prepared from a compound of formula IIf, or a salt thereof, 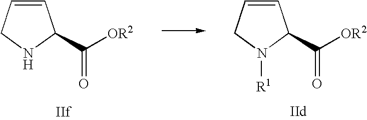
where R² is an alkyl or aryl group and R¹ is as defined in claim 1.

37. A process according to any one of claims 29 to 35 wherein R² is methyl.

38. A process according to any preceding claim wherein R¹ is a Boc group.

39. A process according to any preceding claim which further comprises the step of protecting the free NH group of said compound of formula I.

40. A process according to claim 39 which comprises treating said compound of formula I with Fmoc-Cl and sodium carbonate in 1,4-dioxane/water.

41. A process according to any preceding claim wherein said compound of formula III or IIIa is purified by crystallisation prior to step (ii).

42. A process according to claim 41 wherein said compound of formula IIIa is crystallised from a mixture of diethyl ether: heptane.

43. A process according to any one of claims 1 to 37 or 41 or 42 wherein R¹ is a P₁′ group, and P₁′ is selected from CO-aryl, CO-aralkyl, CO-cycloalkyl, CO-alkyl and CO-alicylic group, wherein said aryl, alkyl, aralkyl, cycloalkyl and alicyclic groups are each optionally substituted by one or more substituents selected from alkyl, alkoxy, halogen, NH₂, CF₃, SO₂-aryl, SO₂-aryl, OH, NH-alkyl, NHCO-alkyl and N(alkyl)₂.

44. A process according to claim 43 wherein said P₁′ group is selected from CO-phenyl, CO—CH₂-phenyl and CO—(N-pyrrolidine) CO-(3-pyridyl) and CO-(3-fluoro-phenyl).

45. A method of preparing a cysteinyl proteinase inhibitor which comprises the process of any one of claims 1 to 44.

46. A method according to claim 45 wherein the cysteinyl proteinase inhibitor is a CAC1 inhibitor.

47. A method according to claim 46 wherein the CAC1 inhibitor is selected from an inhibitor of cathepsin K, cathepsin S, cathepsin F, cathepsin B, cathepsin L, cathepsin V, cathepsin C, falcipain and cruzipain.

48. A method according to claim 47 wherein the CAC1 inhibitor is an inhibitor of cathepsin S.

49. A method according to any one of claims 45 to 48 wherein the cysteinyl proteinase inhibitor is of formula VII 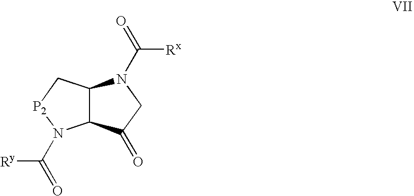
wherein R^{x} and R^{y} are each independently hydrocarbyl.

50. A method according to any one of claims 45 to 49, wherein the cysteinyl proteinase inhibitor is of formula VII 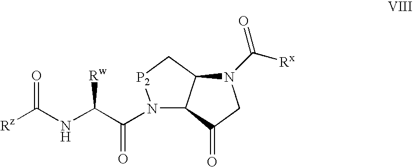
wherein
P₂ is as defined in claim 1;
R^{x} is aryl or alkyl;
R^{w} is alkyl, aralkyl, cycloalkyl(alkyl) or cycloalkyl; and
R^{z} is aryl, heteroaryl or an alicyclic group;
wherein said aryl, alkyl, aralkyl, cycloalkyl(alkyl), cycloalkyl, heteroaryl and alicyclic groups may be optionally substituted.

51. A method according to claim 50 wherein R^{z} is an aryl or heteroaryl group each optionally substituted by a piperazinyl or piperidinyl group, each of which may in turn be optionally substituted by one or more alkyl or acyl groups.

52. A method according to claim 50 wherein R^{z} is a 5-membered heteroaryl group or a 6-membered alicyclic group each optionally substituted by one or more alkyl groups.

53. A method according to claim 50 wherein:
R^{x} is phenyl, 3-pyridyl or 3-fluoro-phenyl;
R^{w} is CH₂CH(Me)₂, cyclohexyl-CH₂—, para-hydroxybenzyl, CH₂C(Me)₃, C(Me)₃, cyclopentyl or cyclohexyl;
R^{z} is phenyl or thienyl each of which may be optionally substituted by one or more substituents selected from OH, halo, alkyl, alkyl-NH₂, N-piperazinyl and N-piperidinyl, wherein said N-piperazinyl and N-piperidinyl are each optionally substituted by one or more alkyl or acyl groups; or R^{z} is 2-furanyl, 3-furanyl or N-morpholinyl each of which may be optionally substituted by one or more alkyl groups.

54. A method according to any one of claims 50 to 53 wherein the cysteinyl proteinase inhibitor is of formula IX 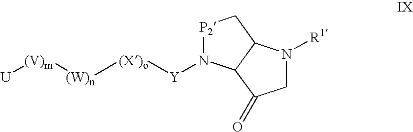
wherein:
P_{2′}═O, CH₂ or NR⁹, where R⁹ is chosen from H, C₁₋₇-alkyl, C₃₋₆-cycloalkyl, Ar or Ar—C₁₋₇-alkyl;
Y═CR¹⁰R¹¹—C(O) or CR¹⁰R¹¹—C(S) or CR¹⁰R¹¹—S(O) or CR¹⁰R¹¹—SO₂ where R¹⁰ and R¹¹ are independently chosen from H, C₁₋₇-alkyl, C₃₋₆-cycloalkyl, Ar and Ar—C₁₋₇-alkyl, or Y represents 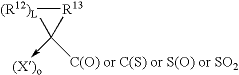
where L is a number from one to four and R¹² and R¹³ are independently chosen from CR¹⁴R¹⁵ where R¹⁴ and R¹⁵ are independently chosen from H, C₁₋₇-alkyl, C₃₋₄-cycloalkyl, Ar, Ar—C₁₋₇-alkyl or halogen; and for each R¹² and R¹³ either R¹⁴ or R⁵ (but not both R¹⁴ and R¹⁵) may additionally be chosen from OH, O—C₁₋₇-alkyl, O—C₃₋₄-cycloalkyl, OAr, O—Ar—C₁₋₇-alkyl, SH, S—C₁₋₇-alkyl, S—C₃₋₆-cycloalkyl, SAr, S—Ar—C₁₋₇-alkyl, NH₂, NH—C₁₋₇-alkyl, NH—C₃₋₆-cycloalkyl, NH—Ar, NH—Ar—C₁₋₇-alkyl, N—(C₁₋₇-alkyl)₂, N—(C₃₋₄-cycloalkyl)₂, NAr₂ and N-(Ar—C₁₋₇-alkyl)₂;
in the group (X′)ₒ, X′═CR¹⁶R¹⁷, where R¹⁶ and R¹⁷ are independently chosen from H, C₁₋₇-alkyl, C₃₋₆-cycloalkyl, Ar and Ar—C₁₋₇-alkyl and o is a number from zero to three;
in the group (W)ₙ, W═O, S, C(O), S(O) or S(O)₂ or NR¹⁸, where R¹⁸ is chosen from H, C₁₋₇-alkyl, C₃₋₆-cycloalkyl, Ar and Ar—C₁₋₇-alkyl and n is zero or one;
in the group (V)ₘ, V═C(O), C(S), S(O), S(O)₂, S(O)₂NH, OC(O), NHC(O), NHS(O), NHS(O)₂, OC(O)NH, C(O)NH or CR¹⁹R²⁰, C═N—C(O)—OR¹⁹ or C═N—C(O)—NHR¹⁹, where R¹⁹ and R²⁰ are independently chosen from H, C₁₋₇-alkyl, C₃₋₆-cycloalkyl, Ar, Ar—C₁₋₁₇-alkyl and m is a number from zero to three, provided that when m is greater than one, (V)ₘ contains a maximum of one carbonyl or sulphonyl group;
U=a stable 5- to 7-membered monocyclic or a stable 8- to 11-membered bicyclic ring which is saturated or unsaturated and which includes zero to four heteroatoms, selected from the following: 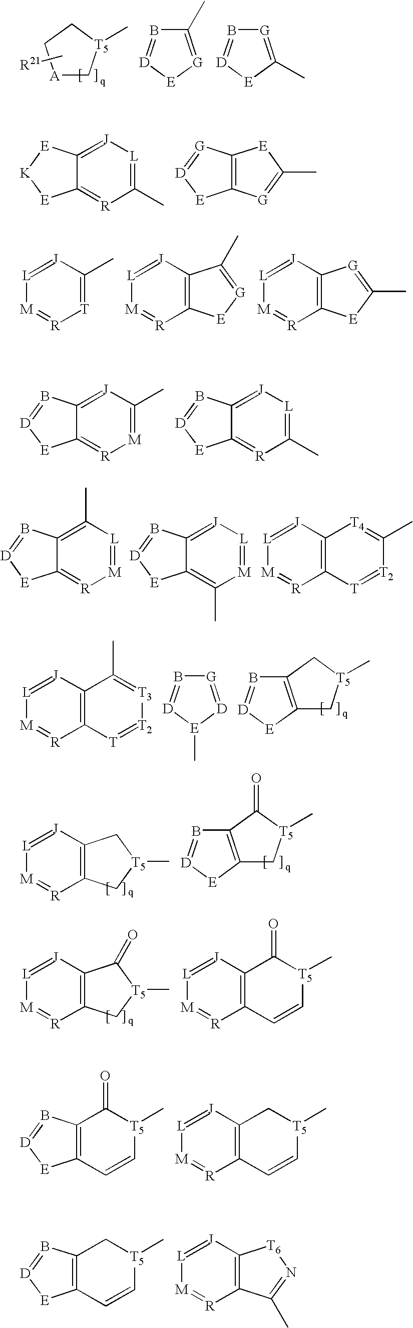 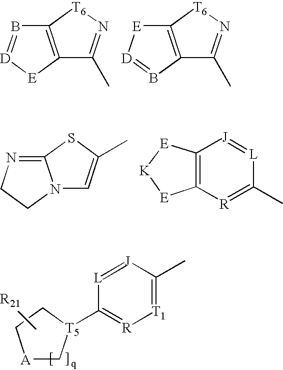
wherein R²¹ is:
H, C₁₋₇-alkyl, C₃₋₆-cycloalkyl, Ar, Ar—C₁₋₇-alkyl, OH, O—C₁₋₇-alkyl, O—C₃₋₆-cycloalkyl, O—Ar, O—Ar—C₁₋₇-alkyl, SH, S—C₁₋₇-alkyl, S—C₃₋₆-cycloalkyl, S—Ar, S—Ar—C₁₋₇-alkyl, SO₂H, SO₂—C₁₋₇-alkyl, SO₂—C₃₋₆-cycloalkyl, SO₂—Ar, SO₂—Ar—C₁₋₇-alkyl, NH₂, NH—C₁₋₇-alkyl, NH—C₃₋₆-cycloalkyl, NH—Ar, N—Ar₂, NH—Ar—C₁₋₇-alkyl, N(C₁₋₇-allyl)₂, N(C₃₋₆-cycloalkyl)₂ or N(Ar—C₁₋₇-alkyl)₂; or, when part of a CHR²¹ or CR²¹ group, R²¹ may be halogen;
A is chosen from:
CH₂, CHR²¹, O, S, SO₂, NR² or N-oxide (N—O), where R²¹ is as defined above; and R²² is chosen from H, C₁₋₇-alkyl, C₃₋₆-cycloalkyl, Ar and Ar—C₁₋₇-alkyl;
B, D and G are independently chosen from:
CR²¹, where R²¹ is as defined above, or N or N-oxide (N→O);
E is chosen from:
CH₂ CHR²¹, O, S, SO₂, NR²² or N-oxide (N→O), where R²¹ and R²² are defined as above;
K is chosen from:
CH₂, CHR²², where R²² is defined as above;
J, L, M, R T, T₂, T₃ and T₄ are independently chosen from:
CR²¹ where R²¹ is as defined above, or N or N-oxide (N→O);
T₅ is chosen from:
CH or N;
T₆ is chosen from:
NR²², SO₂, OC(O), C(O), NR²²C(O);
q is a number from one to three, thereby defining a 5-, 6- or 7-membered ring;
R^{1′}═R^{2′}OC(O), R^{2′}OC(O), R²NQC(O), R^{2′}SO₂, where R^{2′}is chosen from C₁₋₇-alkyl, C₃₋₆-cycloalkyl, Ar and Ar—C₁₋₇-alkyl and Q is H or C₁₋₇-alkyl.

55. A method of preparing a compound of formula VII, VIII or IX as defined in any one of claims 49, 50 or 54, said method comprising the process according to any one of claims 1 to 44.

56. A process or method substantially as described herein with reference to the accompanying examples.
